(19) United States
(12) Patent Application Publication
(10) Pub. No.: US 2018/0161761 A1
(43) Pub. Date: Jun. 14, 2018

(54) ETHYLENE EPOXIDATION CATALYSTS, ASSOCIATED METHODS OF MANUFACTURE, AND ASSOCIATED METHODS FOR THE PRODUCTION OF ETHYLENE OXIDE

(71) Applicant: SHELL OIL COMPANY, Houston, TX (US)

(72) Inventors: Randall Clayton YEATES, Sugar Land, TX (US); John Robert LOCKEMEYER, Sugar Land, TX (US); Donald James REMUS, Fairfax Station, VA (US)

(21) Appl. No.: 15/834,103

(22) Filed: Dec. 7, 2017

Related U.S. Application Data
(60) Provisional application No. 62/432,207, filed on Dec. 9, 2016.

(51) Int. Cl. B01J 23/68; B01J 27/12; B01J 35/00; B01J 35/10; B01J 37/02; C07D 301/10

(52) U.S. Cl. CPC B01J 23/688; B01J 27/12; C07D 301/10; B01J 35/1009; B01J 37/0215; B01J 35/0006

(57) ABSTRACT

An ethylene epoxidation catalyst is disclosed that comprises a fluoride-mineralized carrier having silver and a rhenium promoter deposited thereon, wherein the fluoride-mineralized carrier has: a total fluorine (TF) content less than 5000 ppm as measured by XRF, a water extractable fluorine (WEF) content greater than 45 ppm as measured by microwave extraction and ion specific electrode, and wherein the ratio of TF:WEF is between 10 and 110. Associated methods of manufacturing such catalysts and epoxidation methods using such catalysts are similarly provided.

### CROSS-REFERENCE TO RELATED APPLICATIONS

This application claims the benefit of U.S. Provisional Application No. 62/432,207, filed on Dec. 9, 2016, which is incorporated herein by reference.

### BACKGROUND

Ethylene oxide is a valuable raw material that is well-known for its use as a versatile chemical intermediate in the production of a wide variety of chemicals and products. For example, ethylene oxide is often used to produce ethylene glycol, which is used in many diverse applications and may be found in a variety of products, including automotive engine antifreeze, hydraulic brake fluids, resins, fibers, solvents, paints, plastics, films, household and industrial cleaners, pharmaceutical preparations, and personal care items, such as cosmetics, shampoos, etc.

In the commercial production of ethylene oxide, ethylene is reacted with oxygen in the presence of an epoxidation catalyst, within an epoxidation reactor, to produce a gaseous stream at the outlet of the epoxidation reactor that comprises ethylene oxide. The reactor outlet stream typically comprises, in addition to ethylene oxide, unreacted ethylene, unreacted oxygen, a reaction modifier (e.g., organic chlorides), a dilution gas (e.g., nitrogen, methane or a combination thereof), various by-products of the epoxidation reaction (e.g., carbon dioxide and water) and various impurities (e.g., aldehydes, acidic impurities, argon, ethane, etc.). In a next stage, the ethylene oxide is recovered from the reactor outlet stream, typically by supplying the reactor outlet stream to an ethylene oxide separation system, where the produced ethylene oxide is separated from the majority of the other gaseous constituents through contact with a recirculating solvent (commonly referred to as “lean absorbent”). The produced ethylene oxide is often further reacted, for example to provide glycols (e.g., ethylene glycol, diethylene glycol, triethylene glycol, etc.) via catalytic or non-catalytic hydrolysis. Typically, a majority of the remaining gaseous constituents in the ethylene oxide separation system (e.g., unreacted ethylene, unreacted oxygen, reaction modifier, dilution gas, etc.) are removed therefrom as an overhead gas stream, at least a portion of which is typically recycled to the epoxidation reactor via a recycle gas loop so as to minimize waste and/or increase savings, as the use of a recycle gas stream decreases the amount of fresh “make-up” feed (e.g., ethylene, oxygen, etc.) that needs to be supplied to the epoxidation reactor. Optionally, at least a portion of the recycle gas stream may be supplied to one or more separation and/or purification systems, such as a carbon dioxide separation system, etc., before it is supplied to the epoxidation reactor.

Ethylene oxide is formed by reacting ethylene with oxygen in the presence of a silver-based ethylene epoxidation catalyst. The catalyst performance may be assessed on the basis of selectivity, activity and stability of operation. The selectivity of an ethylene epoxidation catalyst, also known as the “efficiency”, refers to the ability of the epoxidation catalyst to convert ethylene to the desired reaction product, ethylene oxide, versus the competing by-products (e.g., CO₂ and H₂O), and is typically expressed as the percentage of the number of moles of ethylene oxide produced per number of moles of ethylene reacted. Stability refers to how the selectivity and/or activity of the process changes during the time a charge of catalyst is being used, i.e., as more ethylene oxide is produced.

Various approaches to improving the performance of ethylene epoxidation catalysts, including improvements in selectivity, activity, and stability, have been investigated. For example, modern silver-based ethylene epoxidation catalysts may comprise, in addition to silver, a rhenium promoter, and optionally one or more additional promoters, such as alkali metals (e.g., cesium, lithium, etc.), alkaline earth metals (e.g., magnesium), transition metals (e.g., tungsten), and main group non-metals (e.g., sulfur), are disclosed, for example, in U.S. Pat. Nos. 4,761,394 and 4,766,105.

The selectivity determines to a large extent the economical attractiveness of an epoxidation process. For example, one percent improvement in the selectivity of the epoxidation process can substantially reduce the yearly operating costs of a large scale ethylene oxide plant. Further, the longer the activity and selectivity can be maintained at acceptable values, the longer the catalyst charge can be kept in the reactor and the more product is obtained. Quite modest improvements in the selectivity, activity, and maintenance of the selectivity and activity over long periods yield substantial dividends in terms of process efficiency.

Notwithstanding the improvements already achieved, there is a desire to further improve the performance of ethylene epoxidation catalysts.

### SUMMARY

An ethylene epoxidation catalyst is provided. The ethylene epoxidation catalyst comprises a fluoride-mineralized carrier having silver and a rhenium promoter deposited thereon, wherein the fluoride-mineralized carrier has: a total fluorine (TF) content less than 5000 ppm as measured by XRF, a water extractable fluorine (WEF) content greater than 45 ppm as measured by microwave extraction and ion specific electrode, and wherein the ratio of TF:WEF is between 10 and 110.

A method for the epoxidation of ethylene is also provided, which comprises contacting an inlet feed gas comprising ethylene and oxygen with an ethylene epoxidation catalyst comprising a fluoride-mineralized carrier having silver and a rhenium promoter deposited thereon, wherein the fluoride-mineralized carrier has: a total fluorine (TF) content less than 5000 ppm as measured by XRF, a water extractable fluorine (WEF) content greater than 45 ppm as measured by microwave extraction and ion specific electrode, and wherein the ratio of TF:WEF is between 10 and 110.

A method for manufacturing an ethylene epoxidation catalyst is also provided, which comprises depositing silver and a rhenium promoter on a fluoride-mineralized carrier, wherein the fluoride-mineralized carrier has: a total fluorine (TF) content less than 5000 ppm as measured by XRF, a water extractable fluorine (WEF) content greater than 45 ppm as measured by microwave extraction and ion specific electrode, and wherein the ratio of TF:WEF is between 10 and 110.

### DRAWINGS

Some specific example embodiments of the disclosure may be understood by referring, in part, to the following description and the accompanying drawings.

FIG. 1 is a schematic diagram showing an epoxidation process.

FIG. 2 is a Scanning Electron Micrograph (SEM) of a non-fluoride-mineralized carrier's morphology.

FIG. 3 is a SEM of a platelet carrier's morphology.

FIG. 4 is a SEM of a fluoride-mineralized carrier's morphology at 200× magnification.

FIG. 5 is a SEM of a fluoride-mineralized carrier's morphology at 500× magnification.

FIG. 6 is a SEM of a fluoride-mineralized carrier's morphology at 3000× magnification.

FIG. 7 is a SEM of a fluoride-mineralized carrier's morphology at 5000× magnification.

FIG. 8 is the pore size distribution of the fluoride-mineralized carrier shown in FIG. 4.

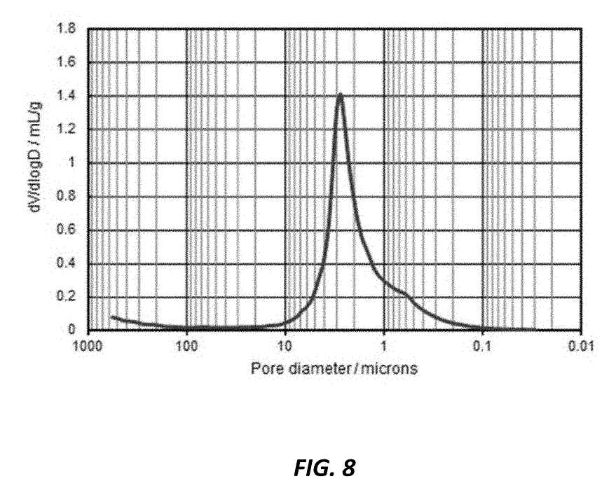

FIG. 9 is a SEM of another fluoride-mineralized carrier's morphology at 200× magnification.

FIG. 10 is the pore size distribution of the fluoride-mineralized carrier shown in FIG. 9.

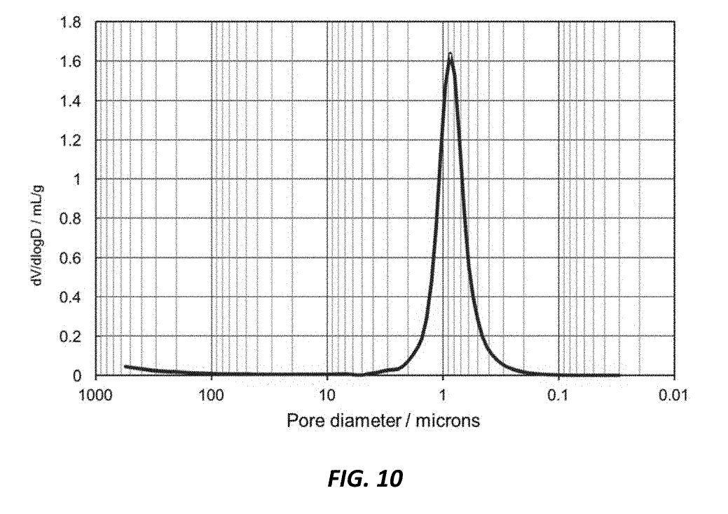

FIG. 11 is a SEM of a fluoride-mineralized carrier at 5000× magnification.

FIG. 12 is a SEM of the same fluoride-mineralized carrier shown in FIG. 11 but at 10,000× magnification.

FIG. 13 is the pore size distribution of the carrier shown in FIGS. 11 and 12.

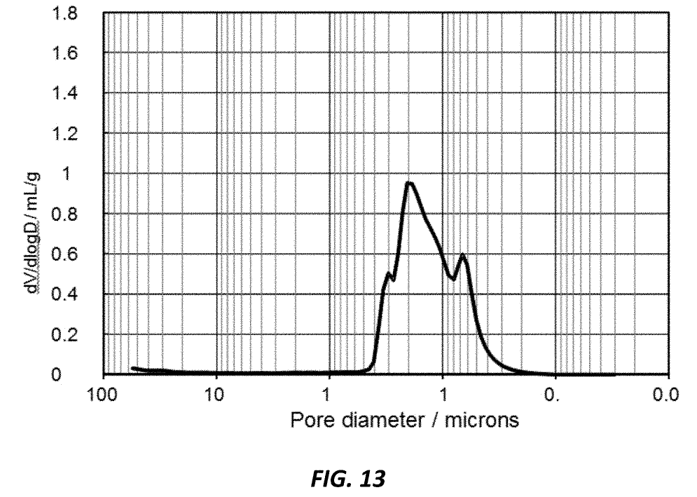

FIG. 14 is a SEM of another fluoride-mineralized carrier at 5000× magnification.

FIG. 15 is the pore size distribution of the fluoride-mineralized carrier shown in FIG. 14.

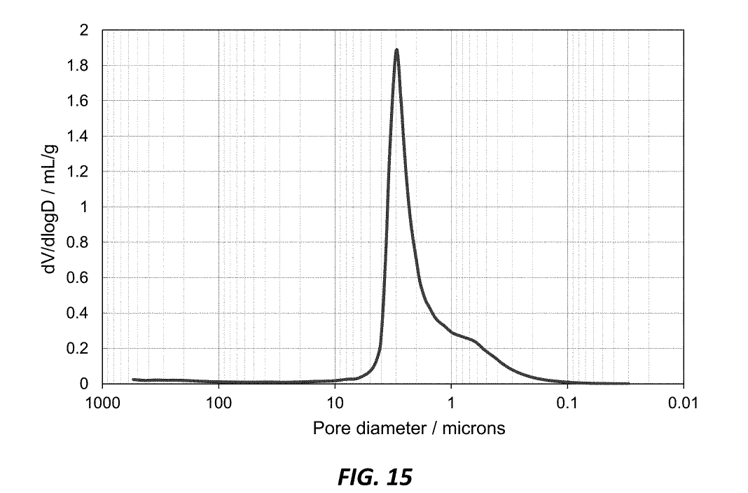

While the present disclosure is susceptible to various modifications and alternative forms, specific example embodiments have been shown in the figures and are herein described in more detail. It should be understood, however, that the description of specific example embodiments is not intended to limit the invention to the particular forms disclosed, but on the contrary, this disclosure is to cover all modifications and equivalents as illustrated, in part, by the appended claims.

### DETAILED DESCRIPTION

To facilitate an understanding of the present disclosure, it is useful to define certain terms relating to the epoxidation reaction and ethylene epoxidation catalyst performance.

As used herein, the “activity” of an ethylene epoxidation catalyst generally refers to the reaction rate of ethylene towards ethylene oxide per unit of epoxidation catalyst volume in the epoxidation reactor and is typically expressed as the temperature required to maintain a given ethylene oxide production rate. In general, the activity of an ethylene epoxidation catalyst is a function of both the total number of catalytically active sites present on the surface of the ethylene epoxidation catalyst and the reaction rate of each site. Thus, the activity of a catalyst will decline if either the number of catalytically active sites on the surface of an epoxidation catalyst is reduced and/or if the reaction rate for one or more of the active sites decreases (e.g., due to localized poisoning). The total number of active sites can be reduced in several ways, for example, by sintering of the catalytically active particles (i.e., silver particles), which results in an increase in silver particle size and correspondingly, a decrease in silver surface area. They can also be reduced by reaction with normal constituents present in the inlet feed gas supplied to the epoxidation reactor, such as by reaction with chloride compounds in the inlet feed gas to form silver chloride compounds, which are inactive towards the epoxidation reaction. Further, the activity can decline due to catalyst poisoning, for example by exposure of the epoxidation catalyst to poisons, such as water or sulfur. In many instances, the production rate of ethylene oxide is described in terms of “work rate”, which refers to the amount of ethylene oxide produced in the epoxidation reactor per hour per unit volume of catalyst (e.g., kilograms or moles of ethylene oxide/hr/m³). As will be appreciated by those skilled in the art, an improvement in the activity of a catalyst, under a given set of conditions, is reflected by a lower reaction temperature required to maintain a given work rate at those conditions. Thus, an ethylene epoxidation catalyst having a “higher activity” in comparison to another ethylene epoxidation catalyst is one that, under a given set of conditions, employs a lower temperature at a given work rate. Alternatively, the activity of an ethylene epoxidation catalyst may be expressed as the mole percent of ethylene oxide contained in the reactor outlet stream relative to that in the inlet feed gas (the mole percent of ethylene oxide in the inlet feed gas typically, but not necessarily, approaches zero percent), while the temperature is maintained substantially constant. Thus, in this instance, an ethylene epoxidation catalyst having a “higher activity” in comparison to another ethylene epoxidation catalyst is one that produces more ethylene oxide at a given temperature under the given set of conditions.

As used herein, the “selectivity” of an ethylene epoxidation catalyst, also known as “efficiency”, refers to the ability of the ethylene epoxidation catalyst to convert ethylene to the desired reaction product, ethylene oxide, versus the competing by-products (e.g., CO₂ and H₂O), and is typically expressed as the percentage of the number of moles of ethylene oxide produced per number of moles of ethylene consumed in the reactor. As will be appreciated by one skilled in the art, an ethylene epoxidation catalyst having a “higher selectivity” in comparison to another ethylene epoxidation catalyst is one that, under a given set of conditions, provides for a greater number of moles of ethylene oxide produced per number of moles of ethylene consumed.

As used herein, “deactivation” refers to a permanent decrease or loss in catalytic activity and/or selectivity. During the epoxidation process, as the ethylene epoxidation catalyst is utilized, the catalyst eventually begins to “age” and its catalytic performance gradually deteriorates (e.g., the activity of the catalyst decreases due to, for example, silver sintering, etc.). Typically, the average useful lifespan of a modern ethylene epoxidation catalyst is approximately two to five years, depending upon factors such as the type of epoxidation catalyst, the temperature, operating conditions, exposure to catalyst poisons, etc. Oftentimes, when catalytic activity begins to decline, the temperature is increased in order to compensate and maintain a constant level of ethylene oxide production (e.g., maintain a desired work rate). However, this increase in temperature often reduces catalyst selectivity and increases the rate of catalyst deactivation (i.e., accelerates the aging of the catalyst). In general, the “stability” of an ethylene epoxidation catalyst is inversely proportional to the rate of catalyst deactivation and is correlative to the length of time that catalyst performance and productivity can be maintained at acceptable values before the catalyst needs to be exchanged for fresh catalyst. As will be readily appreciated, improvements in catalyst stability are highly desirable from an economic perspective because the ethylene epoxidation catalyst is a significant expense to a plant, as is the lost production that occurs due to plant shut down when the catalyst is exchanged.

The present disclosure provides ethylene epoxidation catalysts that comprise a fluoride-mineralized carrier having silver and a rhenium promoter deposited thereon, wherein the fluoride-mineralized carrier has a total fluorine (TF) content less than 5000 parts per million (“ppm”) as measured by x-ray fluorescence (“XRF”), a water extractable fluorine (WEF) content greater than 45 ppm as measured by microwave extraction and ion specific electrode, and wherein the ratio of TF:WEF is between 10 and 110. Associated methods of manufacturing such catalysts and associated methods for the epoxidation of ethylene using such catalysts are similarly provided.

As discussed below, it has been found that ethylene epoxidation catalysts that comprise a fluoride-mineralized carrier having a total fluorine content, water extractable fluorine content and TF:WEF ratio as specified herein may exhibit improved catalyst performance, such as increased selectivity and/or activity, relative to a similar epoxidation catalyst that does not comprise a fluoride-mineralized carrier having the recited features.

A. Epoxidation Process

Although the ethylene epoxidation methods described herein may be carried out in many ways, it is preferred that they be carried out as a gas phase process, i.e. a process in which a feed is contacted in the gas phase with an ethylene epoxidation catalyst which is present as a solid material, typically in a packed bed of a multi-tubular epoxidation reactor. Generally, the ethylene epoxidation process is carried out as a continuous process. The epoxidation reactor is typically equipped with heat exchange facilities to heat or cool the catalyst.

One commercial example of a suitable epoxidation reactor is a vertical shell-and-tube heat exchanger, wherein the shell contains a coolant (e.g., heat transfer fluid (such as tetralin), water, etc.) to regulate the temperature of the epoxidation reactor and wherein the plurality of tubes are substantially parallel, elongated tubes that contain the epoxidation catalyst. While the size and number of tubes may vary from reactor to reactor, a typical tube used in a commercial reactor may have a length of from 3 to 25 meters, from 5 to 20 meters, or from 6 to 15 meters. Similarly, the reactor tubes may have an internal tube diameter of from 5 to 80 millimeters, from 10 to 75 millimeters, or from 20 to 60 millimeters. The number of tubes present in an epoxidation reactor can vary widely and may range in the thousands, for example up to 22,000, or from 1,000 to 11,000, or from 1,500 to 18,500.

The portion of the epoxidation reactor containing the ethylene epoxidation catalyst (e.g., reactor tubes) is commonly referred to as the “catalyst bed”. In general, the amount of ethylene epoxidation catalyst in the catalyst bed, the height of the catalyst bed and the packing density of the ethylene epoxidation catalyst within the catalyst bed (i.e., the “tube packing density”) may vary over a wide range, depending upon, for example, the size and number of tubes present within the epoxidation reactor and the size and shape of the ethylene epoxidation catalyst. However, typical ranges for the tube packing density may be from 400 to 1500 kg/m³. Similarly, typical ranges for catalyst bed height may be from 50% to 100% of the reactor tube length. In those embodiments where the catalyst bed height is less than 100% of the reactor tube length, the remaining portion of the tube may be empty or optionally comprise particles of a non-catalytic or inert material.

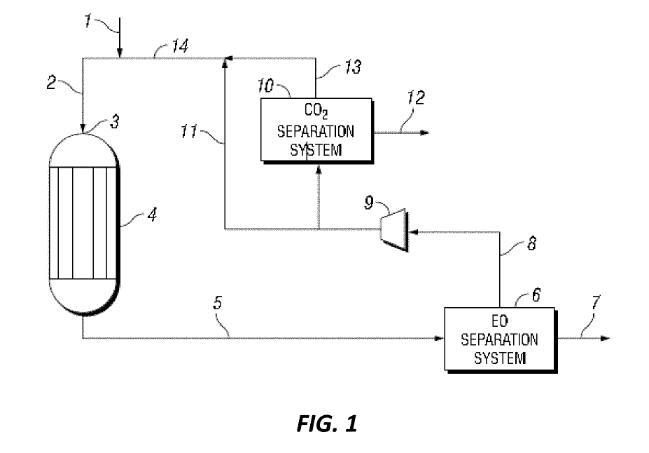

FIG. 1 is a schematic representation showing an exemplary ethylene epoxidation process. Ethylene, oxygen, a dilution gas, and a reaction modifier are supplied at 1 to recycle gas stream 14 to define inlet feed gas 2, which is supplied to inlet 3 of epoxidation reactor 4. Within epoxidation reactor 4, ethylene and oxygen react in the presence of an ethylene epoxidation catalyst. Reactor outlet stream 5, which comprises ethylene oxide, unreacted ethylene, unreacted oxygen, reaction modifier, dilution gas, various by-products of the epoxidation reaction (e.g., carbon dioxide and water) and various impurities, is withdrawn from epoxidation reactor 4 and supplied to ethylene oxide separation system 6. At least a portion of net product stream 7 from ethylene oxide separation system 6 may be further reacted, for example to provide glycols (e.g., ethylene glycol, diethylene glycol, triethylene glycol, etc.) via catalytic or non-catalytic hydrolysis.

A majority of the gaseous constituents not absorbed in ethylene oxide separation system 6 (e.g., unreacted ethylene, unreacted oxygen, reaction modifier, dilution gas, etc.) are withdrawn therefrom as overhead gas stream 8 and supplied to recycle gas compressor 9. At least a portion of overhead gas stream 8 may then be supplied to carbon dioxide separation system 10, while the remaining portion (if any) bypasses the carbon dioxide separation system via bypass stream 11. In carbon dioxide separation system 10, carbon dioxide is removed and exits via carbon dioxide stream 12, while overhead gas stream 13 is combined with bypass stream 11 to form recycle gas stream 14. As previously mentioned, recycle gas stream 14 is combined with “make-up” ethylene, oxygen, dilution gas and reaction modifier to form inlet feed gas 2.

The epoxidation processes described herein are not limited to any particular reactor or flow configurations, and those depicted in FIG. 1 are merely exemplary. Additionally, the sequence in which various feed components are introduced into the process and their respective points of introduction, as well as the flow connections, may be varied from that depicted in FIG. 1.

1. Inlet Feed Gas Composition

In accordance with the epoxidation processes described herein, the inlet feed gas comprises ethylene and oxygen. Optionally, the inlet feed gas may further comprise carbon dioxide, water vapor, a dilution gas, a reaction modifier, and a combination thereof. As used herein, the term “inlet feed gas” is understood to refer to the totality of the gaseous stream at the inlet of the epoxidation reactor. Thus, as will be appreciated by one skilled in the art, the inlet feed gas is often comprised of a combination of one or more gaseous stream(s), such as an ethylene stream, an oxygen stream, a recycle gas stream, etc.

Ethylene may be present in the inlet feed gas in a concentration that may vary over a wide range. However, ethylene is typically present in the inlet feed gas in a concentration of at least 5 mole-%, relative to the total inlet feed gas, or at least 8 mole-%, or at least 10 mole-%, or at least 12 mole-%, or at least 14 mole-%, or at least 20 mole-%, or at least 25 mole-%, on the same basis. Similarly, ethylene is typically present in the inlet feed gas in a concentration of at most 65 mole-%, or at most 60 mole-%, or at most 55 mole-%, or at most 50 mole-%, or at most 48 mole-%, on the same basis. In some embodiments, ethylene may be present in the inlet feed gas in a concentration of from 5 mole-% to 60 mole-%, relative to the total inlet feed gas, or from 10 mole-% to 50 mole-%, or from 12 mole-% to 48 mole-%, on the same basis.

In addition to ethylene, the inlet feed gas further comprises oxygen, which may be provided either as pure oxygen or air. See “Kirk-Othmer Encyclopedia of Chemical Technology”, 3^{rd} edition, Volume 9, 1980, pp. 445-447. In an air-based process, air or air enriched with oxygen is employed, while in an oxygen-based process, high-purity (at least 95 mole-%) oxygen or very high purity (at least 99.5 mole-%) oxygen is employed. Reference may be made to U.S. Pat. No. 6,040,467, incorporated by reference herein, for further description of oxygen-based epoxidation processes. Presently, most epoxidation plants are oxygen-based, which is preferred. Typically, in oxygen-based processes, the inlet feed gas further comprises a dilution gas, which will be discussed in more detail below, to maintain the oxygen concentration below the maximum level allowed by flammability considerations.

In general, the oxygen concentration in the inlet feed gas should be less than the concentration of oxygen that would form a flammable mixture at either the reactor inlet or the reactor outlet at the prevailing operating conditions. Often, in practice, the oxygen concentration in the inlet feed gas may be no greater than a pre-defined percentage (e.g., 95%, 90%, etc.) of oxygen that would form a flammable mixture at either the epoxidation reactor inlet or the epoxidation reactor outlet at the prevailing operating conditions. Although the oxygen concentration may vary over a wide range, the oxygen concentration in the inlet feed gas is typically at least 0.5 mole-%, relative to the total inlet feed gas, or at least 1 mole-%, or at least 2 mole-%, or at least 3 mole-%, or at least 4 mole-%, or at least 5 mole-%, on the same basis. Similarly, the oxygen concentration of the inlet feed gas is typically at most 20 mole-%, relative to the total inlet feed gas, or at most 15 mole-%, or at most 12 mole-%, or at most 10 mole-%, on the same basis. In some embodiments, oxygen may be present in the inlet feed gas in a concentration of from 1 mole-% to 15 mole-%, relative to the total inlet feed gas, or from 2 mole-% to 12 mole-%, or from 3 mole-% to 10 mole-%, on the same basis. Typically, as the oxygen concentration in the inlet feed gas increases, the required operating temperature decreases. However as previously mentioned, in practice, flammability is generally the limiting factor for the maximum concentration of oxygen in the inlet feed gas. Accordingly, in order to remain outside the flammable regime, the oxygen concentration of the inlet feed gas may be lowered as the ethylene concentration of the inlet feed gas is increased. It is within the ability of one skilled in the art to determine a suitable concentration of oxygen to be included in the inlet feed gas, taking into consideration, for example, the overall inlet feed gas composition, along with the other operating conditions, such as pressure and temperature.

Optionally, the inlet feed gas may further comprise carbon dioxide. When present, carbon dioxide is typically present in the inlet feed gas in a concentration of 0.10 mole-% or greater, relative to the total inlet feed gas, or 0.12 mole-% or greater, or 0.15 mole-% or greater, or 0.17 mole-% or greater, or 0.20 mole-% or greater, or 0.22 mole-% or greater, or 0.25 mole-% or greater, on the same basis. Similarly, carbon dioxide is generally present in the inlet feed gas in a concentration of at most 10 mole-%, relative to the total inlet feed gas, or at most 8 mole-%, or at most 5 mole-%, or at most 3 mole-%, or at most 2.5 mole-%, on the same basis. In some embodiments, carbon dioxide may be present in the inlet feed gas in a concentration of from 0.10 mole-% to 10 mole-%, relative to the total inlet feed gas, or from 0.15 mole-% to 5 mole-%, or from 0.20 mole-% to 3 mole-%, or from 0.25 mole-% to 2.5 mole-%, on the same basis. As previously mentioned, carbon dioxide is produced as a reaction by-product and is typically introduced into the inlet feed gas as an impurity (e.g., due to the use of a recycle gas stream in the epoxidation process). Carbon dioxide generally has an adverse effect on catalyst performance, with the operating temperature increasing as the concentration of carbon dioxide present in the inlet feed gas increases. Accordingly, in the commercial production of ethylene oxide, it is common for at least a portion of the carbon dioxide to be continuously removed from the recycle gas stream (e.g., via a carbon dioxide separation system) to maintain the concentration of carbon dioxide in the inlet feed gas at an acceptable level.

Optionally, the inlet feed gas may further comprise water vapor. When present, water vapor is typically present in the inlet feed gas in a concentration such that the partial pressure of water vapor in the inlet feed gas is at least 1 kPA, or at least 2 kPA, or at least 3 kPA, or at least 4 kPA, or at least 5 kPA, or at least 6 kPa, or at least 7 kPa, or at least 8 kPa, or at least 9 kPa, or at least 10 kPa. Similarly, water vapor may be present in the inlet feed gas in a concentration such that the partial pressure of water vapor in the inlet feed gas is at most 1000 kPa, or at most 50 kPa, or at most 40 kPa, or at most 35 kPa, or at most 30 kPa, or at most 25 kPa, or at most 20 kPa, or at most 15 kPa. In some embodiments, water vapor may be present in the inlet feed gas in a concentration such that the partial pressure of water vapor in the inlet feed gas is from 6 to 50 kPa, or from 6 to 40 kPa, or from 6 to 35 kPa, or from 6 to 30 kPa.

As is known to those skilled in the art, the partial pressure of a given gas component in a gaseous mixture may be calculated by multiplying the volume fraction of the gas component that is present in the gaseous mixture by the total absolute pressure exerted by the gaseous mixture. Accordingly, the partial pressure of water vapor in the inlet feed gas may be calculated by multiplying the volume fraction (e.g., mole fraction) of water vapor present in the inlet feed gas by the reactor inlet pressure.

The inlet feed gas optionally may further comprise a dilution gas, such as nitrogen, methane, or a combination thereof. When used, a dilution gas may be added to the inlet feed gas to increase the oxygen flammability concentration. If desired, a dilution gas may be present in the inlet feed gas in a concentration of at least 5 mole-%, relative to the total inlet feed gas, or at least 10 mole-%, or at least 20 mole-%, or at least 25 mole-%, or at least 30 mole-%, on the same basis. Similarly, a dilution gas may be present in the inlet feed gas in a concentration of at most 80 mole-%, relative to the total inlet feed gas, or at most 75 mole-%, or at most 70 mole-%, or at most 65 mole-%, on the same basis. In some embodiments, a dilution gas may be present in the inlet feed gas in a concentration of from 20 mole-% to 80 mole-%, relative to the total inlet feed gas, or from 30 mole-% to 70 mole-%, on the same basis.

Optionally, the inlet feed gas may further comprise a reaction modifier. If desired, a reaction modifier may be added to the inlet feed gas to increase the selectivity of the ethylene epoxidation catalyst. Examples of suitable reaction modifiers may include, but are not limited to, organic chlorides (e.g., C₁ to C₃ chloro hydrocarbons). Specific examples of suitable organic chlorides include, but are not limited to, methyl chloride, ethyl chloride, ethylene dichloride, vinyl chloride, and a combination thereof.

A reaction modifier may optionally be present in the inlet feed gas in a concentration of 0.1 parts per million by volume (ppmv) or greater, relative to the total inlet feed gas, or 0.3 ppmv or greater, or 0.5 ppmv or greater, on the same basis. Similarly, a reaction modifier is generally present in the inlet feed gas in a concentration of at most 25 ppmv, relative to the total inlet feed gas, or at most 22 ppmv, or at most 20 ppmv, on the same basis. In some embodiments, a reaction modifier may be present in the inlet feed gas in a concentration of from 0.1 to 25 ppmv, relative to the total inlet feed gas, or from 0.3 to 20 ppmv, on the same basis. Typically, as the inlet feed gas composition changes and/or as one or more of the operating conditions change, the concentration of reaction modifier in the inlet feed gas may also be adjusted so as to maintain the optimum concentration. For additional disclosure regarding reaction modifiers and optimum concentrations thereof, reference may be made to, for example U.S. Pat. Nos. 7,193,094 and 8,546,592, which are incorporated herein by reference.

Optionally, the inlet feed gas may be substantially free, and preferably completely free, of a nitrogen-containing reaction modifier. That is to say, the inlet feed gas may comprise less than 100 ppm of a nitrogen-containing reaction modifier, preferably less than 10 ppm, more preferably less than 1 ppm, and most preferably 0 ppm of a nitrogen-containing reaction modifier. As used herein, the term “nitrogen-containing reaction modifier” refers to a gaseous compound or volatile liquid that is present as, or capable of forming, nitrogen oxides in oxidizing conditions. Examples of nitrogen-containing reaction modifiers include, but are not limited to, NO, NO₂, N₂O₃, N₂O₄, N₂O₅ or any substance capable of forming one of the aforementioned gases under epoxidation conditions (e.g., hydrazine, hydroxylamine, ammonia, organic nitro compounds (such as nitromethane, nitroethane, nitrobenzene, etc.), amines, amides, organic nitrites (such as methyl nitrite), nitriles (such as acetonitrile)), and a combination thereof.

Furthermore, as previously mentioned, the inlet feed gas may further comprise one or more impurities, such as aldehydes, acidic impurities, argon, ethane, etc. As will be understood by one of skill in the art, the type and concentration of impurities present in the inlet feed gas are determined, at least in part, by the purity of the oxygen and ethylene that is supplied to the epoxidation reactor and the extent to which any such impurities are removed during the epoxidation process.

The order and manner in which the components of the inlet feed gas are combined prior to contacting an ethylene epoxidation catalyst is not limited, and they may be combined simultaneously or sequentially. However, as will be recognized by one skilled in the art, it may be desirable to combine certain components of the inlet feed gas in a specified order for safety reasons. For example, oxygen may be added to the inlet feed gas after the addition of a dilution gas for safety reasons. Similarly, as will be understood by one of skill in the art, the concentration of various feed components present in the inlet feed gas may be adjusted throughout the epoxidation process, for example, to maintain a desired productivity, optimize the epoxidation process, etc. Accordingly, the above-defined concentration ranges were selected to cover the widest possible variations in inlet feed gas composition during normal operation.

2. Operating Conditions

The epoxidation process may be carried out under a broad range of operating conditions that may vary widely between different ethylene oxide plants depending, at least in part, upon the initial plant design, subsequent expansion projects, feedstock availability, the type of ethylene epoxidation catalyst used, process economics, etc. Examples of such operating conditions include, but are not limited to, temperature, reactor inlet pressure, gas flow through the epoxidation reactor (commonly expressed as the gas hourly space velocity or “GHSV”), and the ethylene oxide production rate (commonly described in terms of work rate).

To achieve reasonable commercial ethylene oxide production rates, the epoxidation reaction is typically carried out at a temperature of 180° C. or higher, or 190° C. or higher, or 200° C. or higher, or 210° C. or higher, or 225° C. or higher. Similarly, the temperature is typically 325° C. or lower, or 310° C. or lower, or 300° C. or lower, or 280° C. or lower, or 260° C. or lower. The temperature may be from 180° C. to 325° C., or from 190° C. to 300° C., or from 210° C. to 300° C.

It should be noted that the temperature values used herein refer to the gas phase temperature(s) in the catalyst bed as measured directly through the use of one or more thermocouples. As is known to those of ordinary skill in the art, as a means of monitoring the temperature in a multi-tubular epoxidation reactor, one or more axially positioned thermocouples may be placed in selected reactor tubes. Typically, an epoxidation reactor will contain a total of about 1,000 to about 12,000 reactor tubes, of which reactor tubes between 5 and 50, preferably between 5 and 30 will contain thermocouples. A thermocouple typically runs the entire length of the reactor tube and is usually centered within the tube by one or more positioning devices. Preferably, each thermocouple has 5-10 measurement points along its length (e.g. a multi-point thermocouple) to allow the operator to observe the temperature profile in the catalyst bed. It is within the ability of one skilled in the art to determine which specific reactor tubes within the reactor should contain a thermocouple and where they should be placed so as to permit meaningful and representative measurements. For the sake of accuracy, it is preferred that a plurality of equally spaced thermocouples are utilized, in which case the temperature of a catalyst bed having a relatively uniform loading density would be calculated by taking a numerical average of the plurality of gas temperature measurements, as is known to those of skill in the art.

The epoxidation processes disclosed herein are typically carried out at a reactor inlet pressure of from 1000 to 3000 kPa, or from 1200 to 2500 kPa, absolute. A variety of well-known devices may be used to measure the reactor inlet pressure, for example, pressure-indicating transducers, gauges, etc., may be employed. It is within the ability of one skilled in the art to select a suitable reactor inlet pressure, taking into consideration, for example, the specific type of epoxidation reactor, desired productivity, etc.

The gas flow through the epoxidation reactor is expressed in terms of the Gas Hourly Space Velocity (“GHSV”), which is the quotient of the volumetric flow rate of the inlet feed gas at normal temperature and pressure (e.g., 0° C., 1 atm) divided by the catalyst bed volume (i.e., the volume of the epoxidation reactor that contains epoxidation catalyst). GHSV represents how many times per hour the inlet feed gas would displace the volume of the epoxidation reactor if the gas were at normal temperature and pressure (e.g., 0° C., 1 atm). Generally, as GHSV increases, catalyst selectivity increases. However, for a fixed catalyst volume, increasing GHSV generally leads to increased energy costs; therefore, there is usually an economic trade-off between higher catalyst selectivity and increased operating costs. Typically, in a gas phase epoxidation process, the GHSV is from 1,500 to 10,000 per hour.

As previously mentioned, the production rate of ethylene oxide in an epoxidation reactor is typically described in terms of work rate, which refers to the amount of ethylene oxide produced per hour per unit volume of catalyst. As is known to those skilled in the art, work rate is a function of several different variables, including, but not limited to, temperature, reactor inlet pressure, GHSV, and the composition of the inlet feed gas (e.g., ethylene concentration, oxygen concentration, carbon dioxide concentration, etc.). In general, for a given set of conditions, increasing the temperature at those conditions increases the work rate, resulting in increased ethylene oxide production. However, this increase in temperature often reduces catalyst selectivity and may accelerate the aging of the catalyst. Typically, the work rate in most plants is from 50 to 400 kg of ethylene oxide per m³ of catalyst per hour (kg/m³/h), or from 120 to 350 kg/m³/h.

One skilled in the art with the benefit of the present disclosure will be able to select appropriate operating conditions, such as temperature, reactor inlet pressure, GHSV, and work rate depending upon, for example, plant design, equipment constraints, the inlet feed gas composition, the age of the epoxidation catalyst, etc.

Ethylene oxide produced by the epoxidation processes disclosed herein may be recovered using methods known in the art. In some embodiments, the ethylene oxide may be further reacted with water, an alcohol, carbon dioxide or an amine according to known methods to form ethylene glycol, an ethylene glycol ether, ethylene carbonate or ethanolamine, respectively, if desired.

The conversion into 1,2-ethanediol or the 1,2-ethanediol ether may comprise, for example, reacting ethylene oxide with water, suitably using an acidic or a basic catalyst. For example, for making predominantly 1,2-ethanediol and less 1,2-ethanediol ether, the ethylene oxide may be reacted with a ten-fold molar excess of water, in a liquid phase reaction in presence of an acid catalyst, e.g. 0.5 to 1.0% w sulfuric acid, based on the total reaction mixture, at a temperature of 50° C. to 70° C. and a pressure of 1 bar absolute, or in a gas phase reaction at 130° C. to 240° C. and a pressure of 20 to 40 bar absolute, preferably in the absence of a catalyst. Generally, if the proportion of water is lowered, the proportion of 1,2-ethanediol ethers in the reaction mixture increases. The 1,2-ethanediol ethers thus produced may be a di-ether, tri-ether, tetra-ether or a subsequent ether. Alternative 1,2-ethanediol ethers may be prepared by converting the ethylene oxide with an alcohol, in particular a primary alcohol, such as methanol or ethanol, by replacing at least a portion of the water by the alcohol.

The conversion into ethanolamine may comprise, for example, reacting ethylene oxide with ammonia. Anhydrous or aqueous ammonia may be used, although anhydrous ammonia is typically used to favor the production of monoethanolamine. For methods applicable in the conversion of the ethylene oxide into ethanolamine, reference may be made to, for example, U.S. Pat. No. 4,845,296, which is incorporated herein by reference.

Ethylene glycol and ethylene glycol ether may be used in a large variety of industrial applications, for example in the fields of food, beverages, tobacco, cosmetics, thermoplastic polymers, curable resin systems, detergents, heat transfer systems, etc. Ethylene carbonate may be used as, for example, a precursor in the manufacture of ethylene glycol, or as a diluent, in particular as a solvent. Ethanolamine may be used, for example, in the treating (“sweetening”) of natural gas.

B. Ethylene Epoxidation Catalysts

Ethylene epoxidation catalysts suitable for use in the processes described herein comprise a fluoride-mineralized carrier having silver and a rhenium promoter deposited thereon, wherein the fluoride-mineralized carrier has a total fluorine (TF) content less than 5000 ppm as measured by XRF, a water extractable fluorine (WEF) content greater than 45 ppm as measured by microwave extraction and ion specific electrode, and wherein the ratio of TF:WEF is between 10 and 110. Detailed preparative techniques for fluoride-mineralized carriers and epoxidation catalysts comprising a fluoride-mineralized carrier are provided below.

1. Fluoride-Mineralized Carriers—Preparation/Composition

In general, fluoride-mineralized carriers may be prepared by calcining alpha-alumina precursor(s) in the presence of a fluoride mineralizing agent. The particular manner in which the fluoride-mineralized carrier is prepared is not limited, and therefore any method known in the art for preparing fluoride-mineralized carriers may be used, such as those methods described in U.S. Pat. Nos. 3,950,507, 4,379,134, 4,994,588, 4,994,589 and 6,203,773 and U.S. Patent Pub. No. 2012/0108832, which are incorporated herein by reference, for descriptions relating to the mineralization of alpha-alumina.

One method for preparing a fluoride-mineralized carrier comprises combining alpha-alumina precursors with a fluoride mineralizing agent and calcining the combination. The alpha-alumina precursor(s) may be combined with the fluoride mineralizing agent by any method known in the art. Further, the alpha-alumina precursor(s) and the fluoride mineralizing agent, along with any other desired raw materials, may be provided in any form and combined in any order. For example, alpha-alumina precursor(s) are typically formed into a formed body (e.g., a solid that has been formed into a selected shape suitable for its intended use) and the fluoride mineralizing agent may be combined with the alpha-alumina precursor(s) at any point prior to, during, or after the formation of such formed body and likewise, at any point prior to or during calcination. For example, in some instances, alpha-alumina precursor(s) may be combined with a solution comprising a fluoride mineralizing agent, the combination may be mixed and formed into a formed body (e.g., extruded), and the formed body calcined to form a fluoride-mineralized carrier. Alternatively, alpha-alumina precursor(s) may first be formed into a formed body and then the formed body may be combined with a fluoride mineralizing agent (e.g., by impregnating the formed body with a solution comprising a fluoride mineralizing agent), and subsequently calcined to form a fluoride-mineralized carrier. Furthermore, in other instances, alpha-alumina precursor(s) may be formed into a formed body and then contacted with a fluoride-mineralizing agent during calcination (e.g., by calcining the formed body in a gaseous atmosphere comprising the fluoride mineralizing agent). Accordingly, any known preparative method may be used, provided that the alpha-alumina precursor(s) are calcined in the presence of a fluoride mineralizing agent.

With regards to suitable alpha-alumina precursors, any material that is capable of being at least partially converted to alpha-alumina when heated at a temperature of 1200° C. or less may be used. For example, suitable alpha-alumina precursors include, but are not limited to, aluminum tri-hydroxides, such as gibbsite, bayerite, and nordstrandite; aluminum oxide hydroxides, such as boehmite, pseudo-boehmite and diaspora; transition aluminas, such as gamma-alumina, delta-alumina, eta-alumina, kappa-alumina, chi-alumina, rho-alumina, and theta-alumina; and a combination thereof.

Optionally, suitable fluoride-mineralized carriers may be prepared by calcining a blend of alpha-alumina powder and two alpha-alumina precursors that comprises a first alpha-alumina precursor that has at least 18 weight percent bound water and an amorphous crystalline structure; and a second alpha-alumina precursor that has a primary crystalline structure selected from the group consisting of gamma, chi, delta and theta, and less than 1% by weight of an alpha-alumina crystalline structure. The alpha-alumina powder has a silica content, calculated as SiO₂, of not more than 2000 ppm. In these embodiments, the first alpha-alumina precursor may be present in the blend in a quantity of at most 64% by weight, calculated as the weight of the first alpha-alumina precursor relative to the total combined weight of the alpha-alumina powder and the first and second alpha-alumina precursors. The second alpha-alumina precursor may be present in the blend in a quantity of at least 35% by weight, calculated as the weight of the second alpha-alumina precursor relative to the total combined weight of the alpha-alumina powder and the first and second alpha-alumina precursors. The alpha-alumina powder may be present in the blend in a quantity of at most 2% by weight, calculated as the weight of the alpha-alumina powder relative to the total combined weight of the alpha-alumina powder and the first and second alpha-alumina precursors.

As previously mentioned, the alpha-alumina precursors may be in any form. Typically, alpha-alumina precursor(s) are included in an amount sufficient to provide, after calcination, a fluoride-mineralized carrier that comprises at least 80% by weight, or at least 85% by weight, or at least 90% by weight, or at least 95% by weight alpha-alumina, or up to 99.9% by weight, or to 100% by weight alpha-alumina.

As will be recognized by one skilled in the art, variations in the particulate size(s) of the alpha-alumina powder and the alumina-alumina precursor(s) used has an effect on the physical characteristics of the resulting fluoride-mineralized carrier, such as pore size distribution and total pore volume. Similarly, as will be understood by one of skill in the art, the level of impurities present in a fluoride-mineralized carrier are determined, at least in part, by the purity of the alpha-alumina powder and the alpha-alumina precursor(s) that are used (along with any other raw materials), the degree of volatilization of impurities during calcination, and whether any impurities are removed during any subsequent wash and/or treatment procedures. Common impurities may include silica, alkali and alkaline earth metal oxides and trace amounts of metal and/or non-metal containing additives.

With regards to suitable fluoride mineralizing agents, any material that is volatile or which can be readily volatilized under calcining conditions of the alpha-alumina precursor(s) may be used. Preferably, the fluoride mineralizing agent is capable of providing a volatile fluorine species at a temperature of 1200° C. or less, typically from 800° C. to 1,200° C. Fluoride mineralizing agents may be organic or inorganic and may include ionic, covalent, and polar covalent compounds. The specific form in which a fluoride mineralizing agent is provided is not limited and therefore, a volatile fluorine species may include fluorine, fluoride ions and fluorine-containing compounds. Similarly, the fluoride mineralizing agent may be provided in gaseous or liquid solution (e.g., provided in the form of a solution comprising the fluoride mineralizing agent), or in gaseous form. Examples of suitable fluoride mineralizing agents include, but are not limited to, F₂, aluminum trifluoride (AlF₃), ammonium fluorides, such as ammonium bifluoride (NH₄HF₂) and ammonium fluoride (NH₄F), hydrogen fluoride, hydrofluoric acid, dichlorodifluoromethane (CCl₂F₂), silicon tetrafluoride (SiF₄), silicon hexafluoride ([SiF₆]²⁻), boron trifluoride (BF₃), nitrogen trifluoride (NF₃), xenon difluoride (XeF₂), sulfur hexafluoride (SF₆), phosphorous pentafluoride (PF₅), carbon tetrafluoride (CF₄), fluoroform (CHF₃), tetrafluoroethane (C₂H₂F₄), trifluoroacetic acid, triflic acid, hexafluorosilicates, hexafluorophosphates, tetrafluoroaluminates, alkali (Group 1) fluorides, alkaline earth (Group 2) fluorides, Group 4 fluorides, Group 6 fluorides, Group 8-13 fluorides, lanthanide fluorides, and a combination thereof.

Generally, a fluoride mineralizing agent is used in an amount of at least 0.10% by weight, calculated as the weight of elemental fluorine used relative to the total weight of the alpha-alumina powder and the alpha-alumina precursor(s), and any optional additives, to which the fluoride mineralizing agent is being added. Preferably, the fluoride mineralizing agent is used in an amount no less than 0.20% by weight, more preferably no less than 0.25% by weight. Typically, the fluoride mineralizing agent is used in an amount up to 5% by weight, or up 3% by weight, or up to 2.5% by weight. Although it is possible to use a fluoride mineralizing agent in excess of 5% by weight, such amounts are not generally employed as they are considered unnecessary. These amounts refer to the amount of fluoride mineralizing agent used to prepare a fluoride-mineralized carrier and do not necessarily reflect the amount that may ultimately be present in the fluoride-mineralized carrier, as such amounts will vary depending upon the specific process conditions under which the fluoride-mineralized carrier was made (e.g., calcining temperature, rate of heating, the type and amount of alpha alumina precursor that is used, calcination atmosphere, etc.). Reference is made to, for example, Shaklee, et al, “Growth of α-Al₂O₃ Platelets in the HF-γ-Al₂O₃ System”, Journal of the American Ceramic Society, Volume 77, No. 11 (1994), pp. 2977-2984 for further discussion relating to the effects of fluoride concentration on carrier properties.

If desired, one or more optional additives may be included when preparing a fluoride-mineralized carrier. For example, it may be desirable to include one or more additives to facilitate in forming a formed body and/or to alter one or more of the characteristics of the resulting fluoride-mineralized carrier. Suitable additives may include any of the wide variety of known carrier additives, which include, but are not limited to, bonding agents (e.g., polyolefin oxides, celluloses, alkaline earth metal compounds, such as magnesium silicate and calcium silicate, and alkali metal compounds), extrusion aids (e.g., petroleum jelly, hydrogenated oil, synthetic alcohol, synthetic ester, glycol, starch, polyolefin oxide, polyethylene glycol, and mixtures thereof), solvents (e.g., water), peptizing acids (e.g., an inorganic acid (such as nitric acid), a monofunctional aliphatic carboxylic acid containing from 1 to about 5 carbon atoms (such as acetic acid, propanoic acid and formic acid), a halogenated monofunctional aliphatic carboxylic acid containing from 1 to about 5 carbon atoms (such as mono-, di-, and trichloro acetic acid), etc.), fluxing agents, binders, dispersants, burnout materials (also known as “pore formers”), strength-enhancing additives, etc. It is within the ability of one skilled in the art to select suitable additives in appropriate amounts, taking into consideration, for example, the preparation method and the desired properties of the resulting fluoride-mineralized carrier. Furthermore, alpha-alumina precursor(s) and any other desired additives may be in any form and combined in any order, i.e., the order of addition of alpha-alumina precursor(s) and any other additives may not be critical.

Burnout materials may optionally be included when preparing a fluoride-mineralized carrier to facilitate the shaping of a formed body and/or to alter the porosity of a resulting fluoride-mineralized carrier. Typically, burnout materials are burned out, sublimed, or volatilized during drying or calcining. Examples of suitable burnout materials include, but are not limited to, comminuted shells of nuts such as pecan, cashew, walnut, peach, apricot and filbert, and granulated polyolefins, such as polyethylene and polypropylene.

A strength-enhancing additive may optionally be included in a fluoride-mineralized carrier, for example, to increase the crush strength and/or improve the attrition resistance of the fluoride-mineralized carrier. Reference is made to U.S. Pat. Nos. 7,560,411, 8,513,156, 8,536,083 and 8,603,937, which are incorporated herein by reference, for descriptions relating to strength-enhancing additives. Examples of suitable strength-enhancing additives may include, but are not limited to, a zirconium species, a lanthanide Group species, a Group 2 metal species, an inorganic glass, or mixtures thereof. The specific form in which the strength-enhancing additive exists prior to being incorporated into a fluoride-mineralized carrier is not limited. Thus, a zirconium species, a lanthanide Group species, and a Group 2 metal species includes any specific element as such and compounds of the element. Additionally, the strength-enhancing additive may be used in the form of a composition comprising the strength-enhancing additive, such as a solution or emulsion comprising the strength-enhancing additive. Illustrative strength-enhancing additives include, but are not limited to, ammonium fluorozirconate, calcium zirconate, zirconium acetate, zirconium acetylacetonate, zirconium carbonate, zirconium fluoride, zirconium oxynitrate, zirconium silicate, lanthanum carbonate, lanthanum fluoride, lanthanum nitrate, lanthanum oxalate, lanthanum oxide, cerium carbonate, cerium fluoride, cerium nitrate, cerium oxalate, cerium oxide, magnesium acetate, magnesium carbonate, magnesium fluoride, magnesium nitrate, magnesium oxalate, magnesium oxide, calcium acetate, calcium carbonate, calcium fluoride, calcium nitrate, calcium oxalate, and calcium oxide. In some embodiments, a strength-enhancing additive may be included in an amount of 0.10% by weight to 5% by weight, calculated as the amount of the element used relative to the total weight of alpha-alumina powder and alpha-alumina precursor(s), and any optional additives, to which the strength-enhancing additive is being added.

In those embodiments wherein the strength-enhancing additive comprises inorganic glass, it is preferable that the inorganic glass has a melting temperature that is at most the temperature at which the calcination is carried out. For example, the inorganic glass may have a melting temperature that is below 1,200° C. Melting temperature of the inorganic glass is understood to mean the temperature at which the ingredients of the inorganic glass would be heated during glass manufacture to obtain a fluid. Typical inorganic glass may include the elements silicon, boron, aluminum, or lead in combination with many other elements, such as alkali and alkaline earth metals. These elements are typically employed as their oxides. Illustrative inorganic glass that may be used for purposes of the present disclosure include, among many others, the following: Na₂O·SiO₂+Na₂O·2SiO₂, Na₂O·2SiO₂+SiO₂ (quartz), K₂O·SiO₂+K₂O·2SiO₂, K₂O·2SiO₂+K₂O·4SiO₂, PbO, 2PbO·SiO₂+PbO·SiO₂, Na₂O·SiO₂+Na₂O·2SiO₂+2Na₂O·CaO·3SiO₂, K₂O·2SiO₂+K₂O·2CaO·9SiO₂+K₂O·4SiO₂, Na₂O·4B₂O₃+SiO₂, and Na₂O·2B₂O₃+Na₂O·SiO₂.

Optionally, a potassium compound may be included when preparing a fluoride-mineralized carrier. It may be desirable to include a potassium compound, for example, to form a melt with a low melting point during the calcination. Suitable potassium compounds may include potassium-containing inorganic or organic compounds, such as an inorganic acid salt, an organic acid salt or a hydroxide of potassium and the like, for example, potassium nitrate, potassium nitrite, potassium carbonate, potassium bicarbonate, potassium fluoride, potassium sulfate, potassium stearate, potassium silicate, potassium oxalate, potassium acetate, potassium hydroxide, potassium meta-aluminate. In some embodiments, a potassium compound may be included in an amount of 0.01% by weight to 3% by weight, calculated as the amount of potassium used relative to the total weight of alpha-alumina precursor(s), and any optional additives, to which the potassium compound is being added.

Typically, alpha-alumina precursor(s), and optionally a fluoride mineralizing agent and/or one or more additives, are formed into a formed body prior to calcination. The manner in which a formed body is prepared is not limited and may include any of several known methods. In some embodiments, a formed body may be prepared from a malleable mixture of raw materials comprising alpha-alumina precursor(s), and optionally the fluoride mineralizing agent and/or one or more additives. The malleable mixture of raw materials may be prepared according to any of several known methods (e.g., ball milling, mix-mulling, ribbon blending, vertical screw mixing, V-blending, attrition milling, etc.) and subsequently formed into a formed body by any of several known methods (e.g., extrusion, spraying, spray drying, agglomeration, pressing, injection molding, slip casting, tape casting, roll compaction, etc.). The malleable mixture (e.g., dough, paste, etc.) may be prepared dry (i.e., in the absence of a liquid medium) or wet. For applicable methods, reference may be made to U.S. Pat. Nos. 5,145,824, 5,512,530, 5,384,302, 5,100,859 and 5,733,842, which are herein incorporated by reference.

Once formed, a formed body may optionally be heated under an atmosphere sufficient to remove water, decompose any organic additives, or otherwise modify the formed body prior to calcination. Suitable atmospheres include, but are not limited to, air, nitrogen, argon, hydrogen, carbon dioxide, water vapor, those comprising fluorine-containing gases or combinations thereof. If desired, such heating is generally conducted at a temperature in the range of from 20° C. to 500° C. and preferably between 30° C. and 300° C., typically for a period of time of at least one minute up to 100 hours and preferably from 5 minutes to 50 hours. Vessels suitable for drying are generally known in the art and may be the same or different than the vessel used for calcination.

Calcination is generally conducted at a temperature that is high enough, and for a period of time that is sufficiently long enough, to induce mineralization of at least a portion of the alpha-alumina precursor(s). In particular, calcination may be conducted at one or more temperatures, at one or more pressures, and for one or more time periods, sufficient to convert at least 50%, or at least 75%, or at least 85%, or at least 90% or at least 95% of the alpha-alumina precursor(s) to alpha-alumina. Calcining may be carried out in any suitable atmosphere, including but not limited to, air, nitrogen, argon, helium, carbon dioxide, water vapor, those comprising a fluoride mineralizing agent and a combination thereof. However, in those embodiments where a formed body further comprises an organic burnout material, at least one of heating and/or calcining is at least partially or entirely carried out in an oxidizing atmosphere, such as in an oxygen-containing atmosphere.

Calcining generally occurs at a temperature of 1200° C. or less, and preferably occurs at a temperature of 750° C. or greater, and even more preferably at a temperature of 900° C. or greater. It is generally desirable to maintain the calcination temperature at 1200° C. or less to prevent excessive amounts of fluoride from being liberated, as this may have a detrimental effect on the morphology of the fluoride-mineralized carrier. The pressure during calcination may be any pressure, including sub atmospheric, atmospheric and super atmospheric pressure. Preferably, calcination is conducted at atmospheric pressure. Depending upon the calcination temperature, calcining typically occurs for a period of time of up to 5 hours, preferably from 0.5 to 3 hours, at atmospheric pressure. As would be recognized by one skilled in the art, if calcining is conducted at a lower temperature, a longer period of time is generally required for the mineralization process and likewise, if calcining is conducted at a higher temperature, the mineralization process typically requires less time.

While it is provided herein that calcination should generally be conducted at a temperature that is high enough, and for a period of time that is sufficiently long enough, to induce mineralization of at least a portion of the alpha-alumina precursor(s) (e.g., at a temperature in a range of from 750° C. to 1200° C., for a period of time from 0.5 to 3 hours, and at atmospheric pressure), the present disclosure is nevertheless independent of the manner by which calcination is conducted. Thus, variations in calcining known in the art, such as holding at one temperature for a certain period of time and then raising the temperature to a second temperature over the course of a second period of time, are contemplated by the present disclosure. Similarly, it should be noted that the surface properties of the resulting fluoride-mineralized carrier depend not only on the calcining temperature but also, at least in part, on the rate of heating during calcination. It is within the ability of one skilled in the art to select suitable calcination conditions, taking into consideration, for example, the desired properties of the resulting fluoride-mineralized carrier. Reference is made to, for example, U.S. Pat. No. 4,379,134, and Daimon, et al., “Morphology of Corundum Crystallized by Heating Mixture of η-Al₂O₃ and AlF₃”, Journal of Crystal Growth, Volume 75 (1986), pp. 348-352 for further discussion relating to the effects of temperature on the mineralization process.

With respect to suitable vessels for calcining, such vessels are generally known in the art. The specific vessel in which calcining is performed is not limited, and therefore any suitable vessel known in the art may be used. Examples of such vessels include, but are not limited to, furnaces, such as a static kiln, a rotary kiln, etc. Furthermore, the temperature and pressure within such vessel may be measured by any suitable means.

After calcining, the resulting fluoride-mineralized carrier may optionally be washed and/or treated prior to deposition of the catalytic material (e.g., silver). Likewise, if desired, any raw materials used to form the fluoride-mineralized carrier may be washed and/or treated prior to calcination. Any method known in the art for washing and/or treating may be used in accordance with the present disclosure, provided that such method does not negatively affect the performance of the resulting epoxidation catalyst. Reference is made to U.S. Pat. Nos. 6,368,998, 7,232,918 and 7,741,499 which are incorporated herein by reference, for descriptions relating to such methods. If washing is desired, it is typically conducted at a temperature in the range of from 15° C. to 120° C. and for a period of time up to 100 hours and preferably from 5 minutes to 50 hours. Washing may be conducted in either a continuous or batch fashion.

Examples of suitable washing solutions may include, but are not limited, water (e.g., deionized water), aqueous solutions comprising one or more salts (e.g., ammonium salts), amine solutions (e.g., ethylenediamine), aqueous organic diluents and a combination thereof. Similarly, suitable aqueous solutions may be acidic, basic or neutral. The volume of washing solution may be such that the fluoride-mineralized carrier is impregnated until a point of incipient wetness of the carrier has been reached. Alternatively, a larger volume may be used and the surplus of solution may be removed from the wet carrier, for example, by centrifugation. Furthermore, following any washing and/or treating step, it is preferable, prior to deposition of the catalytic material (e.g., silver), to dry or roast the fluoride-mineralized carrier. For example, the carrier may be dried in a stream of air, for example at a temperature of from 80° C. to 400° C., for a sufficient period of time.

Processes to manufacture carrier that incorporate the use of a mineralizing agent that includes a fluorine containing compound have been described in numerous publications including: U.S. Pat. No. 4,379,134: U.S. Pat. No. 8,513,156; U.S. Pat. No. 8,546,294 and U.S. Pat. No. 8,685,883. More specifically, in U.S. Pat. No. 4,379,134, which was granted in 1983, a “typical” process is described in column 6 beginning at line 9. The typical process involves a single step calcination which is done in a direct fired rotary calciner. The process has a steep temperature gradient at the feed end of the calciner. To minimize any breakage problems that may be caused by the steep temperature gradient a two step calcination process is disclosed in column 6 beginning at line 17. The use of a rotary kiln furnace, including a direct-fired rotary kiln furnace, as part of the two step process is described in column 9 beginning at line 24 and again in column 10 beginning at line 44. Temperature ranges and other processing parameters for the first and second steps are disclosed.

2. Fluoride-Mineralized Carrier—Chemical and Physical Properties

As previously mentioned, fluoride-mineralized carriers suitable for use herein have a total fluorine (TF) content less than 5000 ppm as measured by XRF, a water extractable fluorine (WEF) content greater than 45 ppm as measured by microwave extraction and ion specific electrode, wherein the ratio of TF:WEF is between 10 and 110.

The total fluorine (TF) content of a fluoride-mineralized carrier suitable for use herein is at most 5000 ppm, or at most 4500 ppm, or at most 4000 ppm, or at most 3500. Similarly, the total fluorine (TF) content of a fluoride-mineralized carrier suitable for use herein may be at least 200 ppm, or at least 1000 ppm, or at least 2000 ppm, or at least 2500 ppm. Further, the total fluorine (TF) content of a fluoride-mineralized carrier suitable for use herein may be from 200 to 5000 ppm, or from 1000 to 5000 ppm, or from 2500 to 4500 ppm. As used herein, “total fluorine content” is understood to refer to the total fluorine content of the fluoride-mineralized carrier as measured by x-ray fluorescence using the following process. First, a fused disc that measures approximately 3 cm wide and 4 mm thick is made as follows. Use a mortar and pestle to grind slightly more than 10 grams of the carrier. Then weigh 10.00+/−0.01 grams of the ground carrier into a plastic container and add 1.00+/−0.01 grams of Licowax® C Micropowder Binder, which is available from Clariant in Charlotte, N.C. USA. Insert into a SPEX® CertiPrep Mixer/Mill® (available from SPEX CertiPrep in Metuchen, N.J. USA) the ground carrier and binder and the mixer's plastic ball for one minute and thirty seconds. Transfer the milled powder to the SPEX® press pellet cup. Put the pellet cup into the die and insert the SPEX® CertiPrep tungsten-carbide pellet with the polished side toward the sample. Press the pellet using the SPEX® 3630 X-press by pressing to thirty tons and holding for three minutes and releasing for two minutes. The chemical composition of the disc is then determined using a model Panalytical MagiX Pro X-Ray Fluorescence analyzer running Super Q software. The water extractable fluorine (WEF) content of a fluoride-mineralized carrier suitable for use herein is at least 45 ppm, or at least 50 ppm, or at least 55 ppm, or at least 60 ppm. Similarly, the total fluorine (TF) content of a fluoride-mineralized carrier suitable for use herein may be at most 300 ppm, or at most 250 ppm, or at most 225 ppm, or at most 200. Further, the water extractable fluorine (WEF) content of a fluoride-mineralized carrier suitable for use herein may be from 45 to 300 ppm, or from 50 to 250 ppm, or from 50 to 225 ppm, or from 50 to 200 ppm. As used herein, “water extractable fluorine content” is understood to refer to the water extractable fluorine content of the fluoride-mineralized carrier as measured by microwave extraction and ion specific electrode. Measurement of the fluoride content in the fluoride-mineralized carrier is accomplished in two steps. The first step is the extraction procedure which generates a leachate. The second step uses an ion selective electrode to measure the ion concentration in the leachate. The ppm of fluorine on the carrier can then be calculated.

With regard to the extraction procedure, begin by weighing 5.00 grams+/−0.05 grams of the fluoride-mineralized carrier into a Teflon® vessel. Add 50 ml+/−1.0 ml of deionized water to the vessel. Load the vessel into a CEM MARS 5 Microwave System which is available from CEM Corporation in Matthews, N.C. USA. Using the heating program set the following operating parameters and start the leaching program: Stage 1; max power 800 W; percent power 100; ramp time in minutes 25:00; pressure (psi) 0350; temperature (C) 115—controlling; hold (min) 20:00. When the leaching program ends leave the vessel in the MARS 5 to cool for thirty minutes or until the screen temperature is less than or equal to 25° C. Place a clean and dry 120 ml polyethylene bottle on an analytical balance and tare. Transfer the contents from the vessel to the tared bottle by rinsing the vessel three times with approximately one to two milliliters of deionized water. Weigh the plastic bottle and contents to determine the final volume of solution (1 g=1 ml). The solution volume equals the total weight of liquid and sample minus the initial weight of the sample. Cap the bottle and shake thoroughly. Filter the solution through Whatman #40 filter (available from GE Healthcare Bio-Sciences in Pittsburgh, Pa., USA) using plastic funnels and plastic 50 ml cups and lids or centrifuge tubes thereby generating the leachate that contains the water extractable fluorine.

Turning now to the process for measuring the fluorine concentration in the solution, begin by using a fluoride ion selective electrode (ISEF12101 from Hach Company of Loveland, Colo., USA) and standard solutions to calibrate the pH meter. Then pipette 25 ml of the leachate into a 50 ml plastic beaker. Add the contents of one TISAB fluoride adjustment buffer powder pillow from Hach Company. Use a stir bar to mix and then let stand for two hours to equilibrate. Cover the solution to avoid evaporation. Rinse the fluorine ion selective electrode with deionized water and blot dry with a lint free cloth. Place the beaker on an electromagnetic stirrer and stir at a moderate rate. Read the concentration when the display on the pH meter (HQd meter from Hach Company) stabilizes. If the reading exceeds 10 ppm then dilute the sample with 25 ml of deionized water, stir well and read again.

The concentration of water extractable fluorine on the carrier can be calculated using the following formula.

ppm=(C×V)/W

wherein C=concentration of the element

W=sample weight, grams

V=sample volume which equals the total weight of liquid and sample minus the initial weight of sample.

Further, fluoride-mineralized carriers suitable for use herein have a total fluorine (TF) content and a water extractable fluorine (WEF) content such that the ratio of the total fluorine (TF) content to the water extractable fluorine (WEF) content (“TF:WEF”) is between 10 and 110, or between 15 and 90, or between 15 and 70.

Fluoride-mineralized carriers suitable for use herein may be selected from those having a varied and wide range of physical properties, including shape, size, packing density, surface area, water absorption, crush strength, attrition resistance, total pore volume, median pore diameter, pore size distributions, morphology, etc.

Suitable shapes for a fluoride-mineralized carrier include any of the wide variety of shapes known for carriers, which include, but are not limited to, pills, chunks, tablets, pieces, pellets, rings, spheres, wagon wheels, trapezoidal bodies, doughnuts, amphora, rings, Raschig rings, honeycombs, monoliths, saddles, cylinders, hollow cylinders, multi-lobed cylinders, cross-partitioned hollow cylinders (e.g., cylinders having at least one partition extending between walls), cylinders having gas channels from side wall to side wall, cylinders having two or more gas channels, and ribbed or finned structures. While the cylinders are often circular, other cross-sections, such as oval, hexagonal, quadrilateral, trilateral, and multi-lobed may be useful. Reference may be made to U.S. Patent Pub. No. 2012/0171407 incorporated by reference herein, for further description of multi-lobed carriers.

Additionally, the size of the fluoride-mineralized carrier is generally not limited, and may include any size suitable for use in an epoxidation reactor. For example, a fluoride-mineralized carrier may be in the shape of a cylinder having a length of 5 to 15 millimeters (“mm”), an outside diameter of 5 to 15 mm, and an inside diameter of 0.2 to 4 mm. In some embodiments, the fluoride-mineralized carrier may have a length-to-outside diameter ratio of 0.8 to 1.2. Additionally, the fluoride-mineralized carrier may be in the shape of a hollow cylinder with a wall thickness of 1 to 7 mm. It is within the ability of one skilled in the art, with the benefit of this disclosure, to select a suitable shape and size of a fluoride-mineralized carrier, taking into consideration, for example, the type and configuration of the epoxidation reactor in which the fluoride-mineralized carrier will be employed (e.g., the length and internal diameter of the tubes within the epoxidation reactor).

In general, the surface area of a carrier is indicative of the amount of surface area per gram of carrier that is available for the deposition of catalytic material (e.g., silver). The surface area of a fluoride-mineralized carrier suitable for use herein is not narrowly critical and may be, for example, from 0.1 to 10 m²/g, relative to the weight of the fluoride-mineralized carrier, or from 0.5 to 5 m²/g, or from 0.7 to 3 m²/g, or at least 0.1 m²/g, or at least 0.3 m²/g, or at least 0.5 m²/g, or at least 0.6 m²/g, or at most 10 m²/g, or at most 5 m²/g, or at most 3 m²/g, on the same basis. As used herein, “surface area” is understood to refer to the surface area of the fluoride-mineralized carrier as measured in accordance with the B.E.T. (Brunauer, Emmett and Teller) method as described in detail in Brunauer, S., Emmet, P. Y. and Teller, E., J. Am. Chem. Soc., 60, 309-16 (1938).

The water absorption of a carrier is typically expressed as the weight of water than can be absorbed into the pores of the carrier, relative to the weight of the carrier, and therefore reported as grams of water per gram of carrier and the units may be abbreviated as “g/g”. Typically, the water absorption of a fluoride-mineralized carrier suitable for use herein may be, for example, from 0.2 to 1.2 g/g, relative to the weight of the fluoride-mineralized carrier, or from 0.3 g/g, or at least 0.2 g/g, or at least 0.3 g/g, or at most 0.8 g/g, or at most 0.7 g/g, on the same basis. As used herein, the term “water absorption” is understood to refer to the water absorption of a carrier as measured in accordance with the following procedure: First, approximately 100 g of representative samples of fluoride-mineralized carrier are dried at 110° C. for a minimum of one hour. The samples are then cooled in a desiccator and the dry weight (D) of each sample is then determined to the nearest 0.01 g. The samples are then placed in a pan of distilled water and boiled for thirty minutes. While the water is boiling, the samples are covered with water and setter pins or some similar device are used to separate the samples from the bottom and sides of the pan and from each other. After the thirty minute boil, the samples are transferred to room temperature water and allowed to soak for an additional fifteen minutes. After returning to room temperature, each sample is then blotted lightly with a moistened, lint-free linen or cotton cloth to remove all excess water from the surface and the saturated weight (M) of each sample is determined to the nearest 0.01 g. The blotting operation may be accomplished by rolling the specimen lightly on the wet cloth which shall previously have been saturated with water and then pressed only enough to remove such water as will drip from the cloth. Excessive blotting should be avoided because it will introduce error by withdrawing water from the pores of the sample. The samples should be weighed immediately after blotting. The entire operation should be completed as quickly as possible to minimize errors caused by evaporation of water from the sample. Water absorption (A) is expressed as the weight of water absorbed, relative to the weight of the dried carrier and is determined using the following formula: A=[(M−D)/D] wherein the water absorption is expressed in units of grams of water per gram of carrier (“g/g”). Water absorption may also be expressed in units of “cc/g”, provided there is a correction for the density of water at the conditions measured. Alternatively, when water absorption is measured according to the above described procedure, it may be convenient to express the water absorption in units of grams of water absorbed per 100 grams of carrier (e.g., 60 g/100 g), which may also be expressed as the weight percentage of water absorbed per 100 g of carrier (e.g., 60%). The water absorption of a carrier may be positively correlated to and thus used interchangeably with the term “porosity” which, in the field of catalyst carriers, is usually understood to mean the carrier's open cell porosity. Generally, as the water absorption of a carrier increases, the ease of deposition of catalytic material on the carrier increases. However, at higher water absorptions, the fluoride-mineralized carrier, or an epoxidation catalyst comprising the carrier, may have lower crush strength or attrition resistance.

The crush strength of a carrier is typically expressed as the amount of compressive force required to crush the carrier, relative to the length of the carrier, and therefore reported as the amount of force per millimeter of carrier and the units may be abbreviated as “N/mm”. The crush strength of a fluoride-mineralized carrier suitable for use herein is not narrowly critical, although it should have a crush strength sufficient to allow for its use in the commercial production of ethylene oxide. Typically, the crush strength of a fluoride-mineralized carrier suitable for use herein may be, for example, at least 1.8 N/mm, or at least 2 N/mm, or at least 3.5 N/mm, or at least 5 N/mm and frequently as much as 40 N/mm, or as much as 25 N/mm, or as much as 15 N/mm. As used herein, the term “crush strength” is understood to refer to the crush strength of a carrier as measured in accordance with ASTM D6175-03, wherein the test sample is tested as such after its preparation, that is with elimination of Step 7.2 of said method, which represents a step of drying the test sample. For this crush strength test method, the crush strength of the carrier is typically measured as the crush strength of hollow cylindrical particles of 8.8 mm external diameter, 3.5 mm internal diameter, and 8 mm length.

In general, the attrition resistance of a carrier is indicative of the propensity of the carrier to produce fines in the course of transportation, handing and use. The attrition resistance of a fluoride-mineralized carrier suitable for use herein is not narrowly critical, although it should be sufficiently robust so to allow for its use in the commercial production of ethylene oxide. Typically, a fluoride-mineralized carrier suitable for use herein may exhibit an attrition of at most 50%, or at most 40%, or at most 30% and is typically at least 5%, or at least 10%, or at least 15%, or at least 20%. As used herein, “attrition resistance” is understood to refer to the attrition resistance of a carrier as measured in accordance with ASTM D4058-92, wherein the test sample is tested as such after its preparation, that is with elimination of Step 6.4 of the said method, which represents a step of drying the test sample. For this test method, the attrition resistance of the carrier is typically measured as the attrition resistance of hollow cylindrical particles of 8.8 mm external diameter, 3.5 mm internal diameter, and 8 mm length.

The total pore volume, the median pore diameter, and the pore size distribution of a carrier may be measured by a conventional mercury intrusion porosimetry device in which liquid mercury is forced into the pores of a carrier. Greater pressure is needed to force the mercury into the smaller pores and the measurement of pressure increments corresponds to volume increments in the pores penetrated and hence to the size of the pores in the incremental volume. As used herein, the pore size distribution, the median pore diameter and the pore volumes are as measured by mercury intrusion porosimetry to a pressure of 2.1×10⁸ Pa using a Micromeritics Autopore 9200 model (130° contact angle, mercury with a surface tension of 0.480 N/m, and correction for mercury compression applied). As used herein, the median pore diameter is understood to mean the pore diameter corresponding to the point in the pore size distribution at which 50% of the total pore volume is found in pores having less than (or greater than) said point.

The total pore volume of a fluoride-mineralized carrier suitable for use herein is not narrowly critical and may be, for example, at least 0.20 mL/g, at least 0.30 mL/g, at least 0.40 mL/g, at least 0.50 mL/g and is typically at most 0.80 mL/g, at most 0.75 mL/g, or at most 0.70 mL/g. Generally, as the total pore volume of a carrier increases, the ability to deposit catalytic material on the carrier increases. However, at higher total pore volumes, the fluoride-mineralized carrier, or an epoxidation catalyst comprising the carrier, may have lower crush strength or attrition resistance. The median pore diameter of a fluoride-mineralized carrier suitable for use herein is not narrowly critical and may be, for example, from 0.50 to 50 μm. In addition, fluoride-mineralized carriers suitable for use herein may have a pore size distribution that is monomodal, bimodal or multimodal.

A fluoride-mineralized carrier suitable for use herein may have, and preferably does have, a particulate matrix having a morphology characterizable as platelet-type. As such, particles having in at least one direction a size greater than 0.1 micrometers have at least one substantially flat major surface. Such particles may have two or more flat major surfaces.

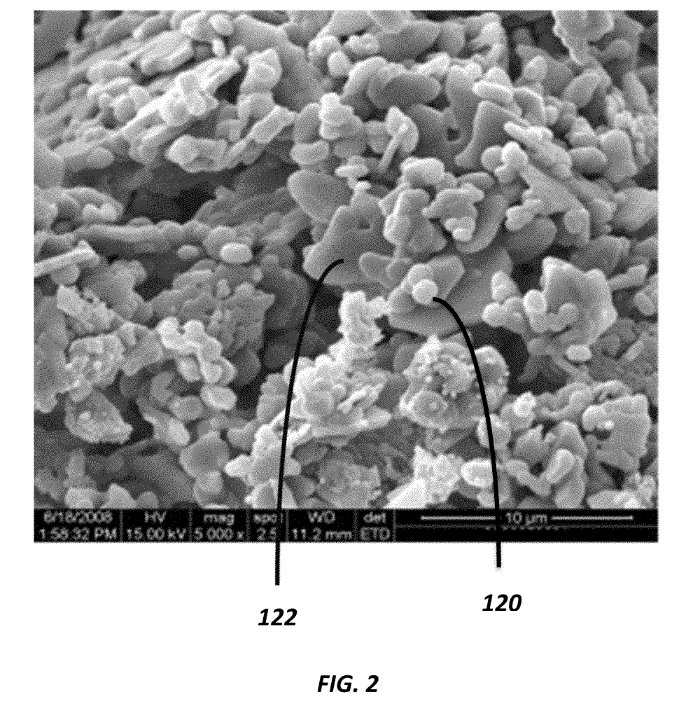

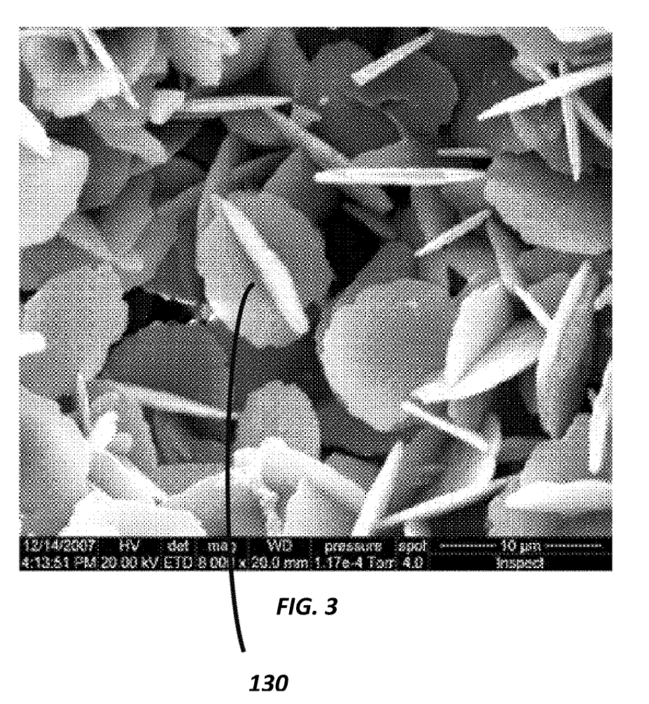

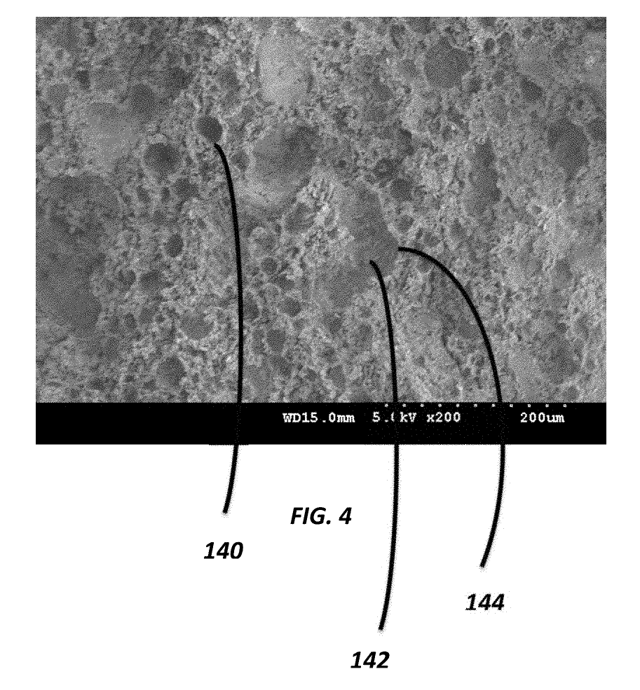

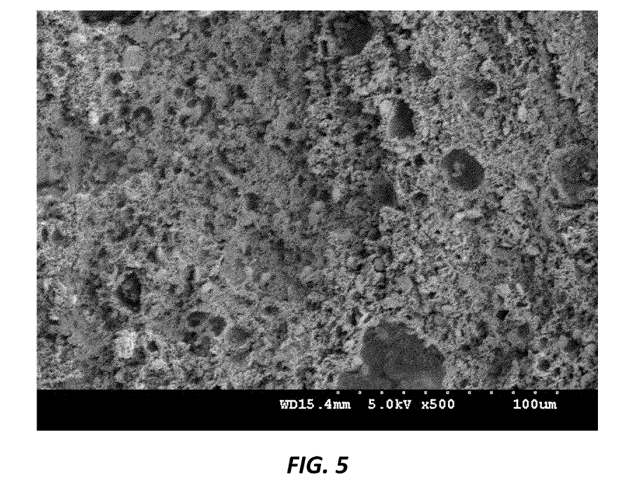

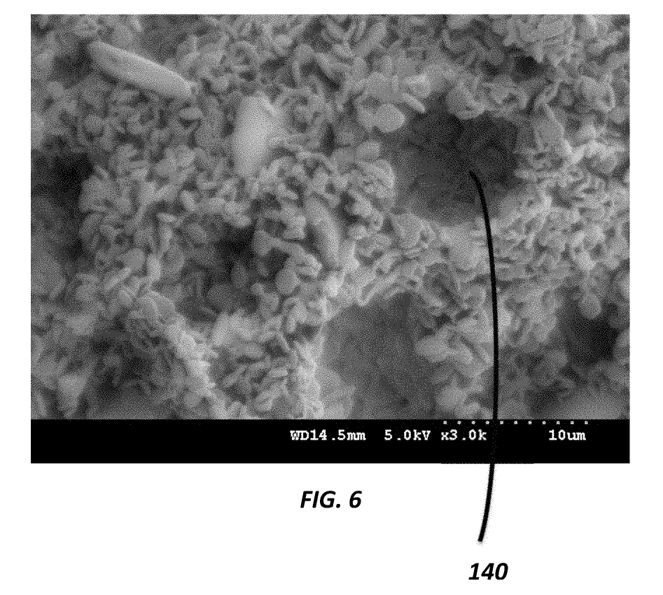

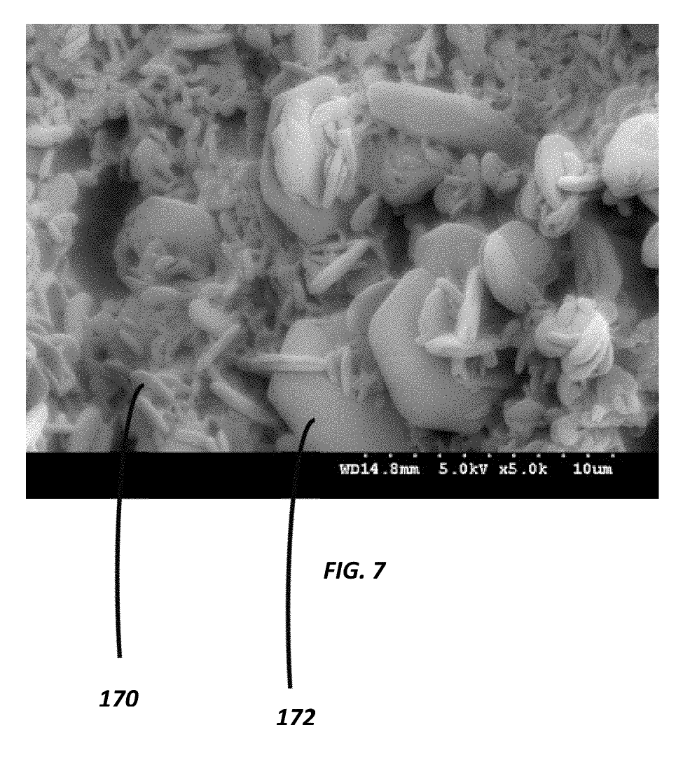

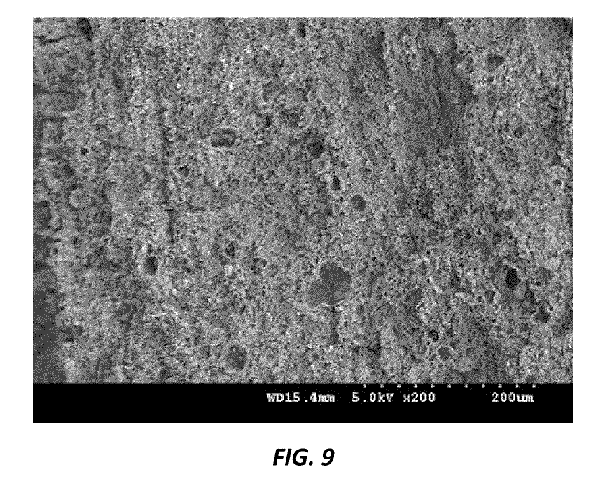

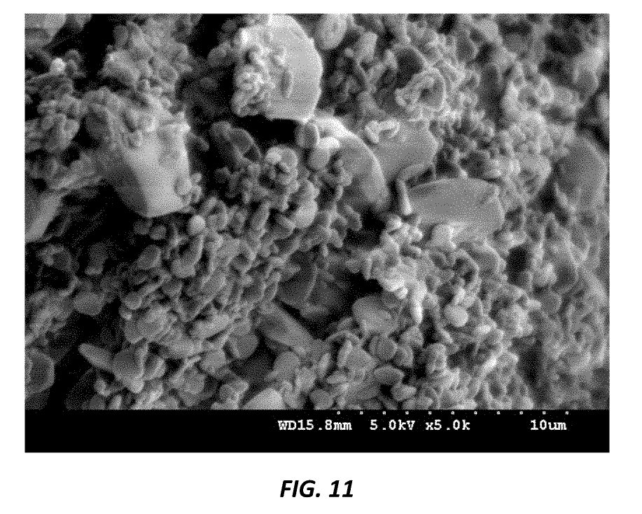

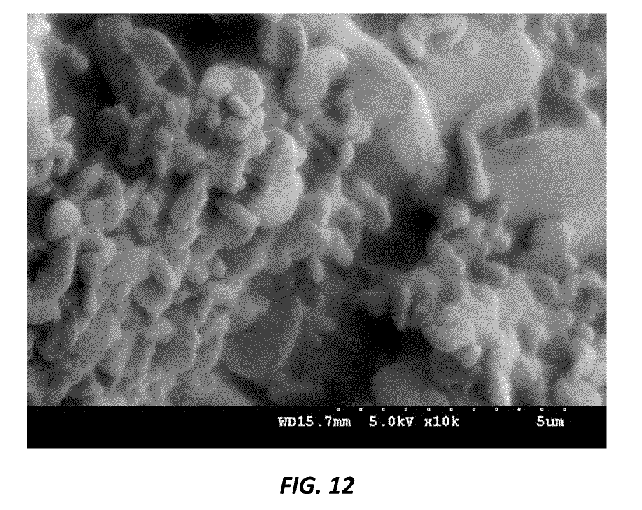

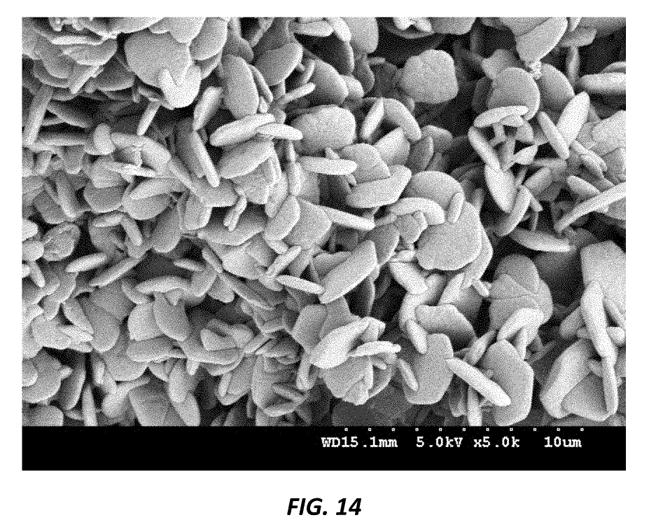

Reference is made to FIGS. 4-7, 9, 11, 12 and 14 which are Scanning Electron Micrographs (SEMs) that show the morphology of carriers used in the epoxidation of ethylene. FIG. 2 is a SEM at 5000× magnification of a non-fluoride-mineralized carrier, which may be referred to herein as a “conventional” carrier. This non-fluoride-mineralized carrier comprises a plurality of granular particles 120 that appear to rest on the surface of the non-interlocking planar components 122. Shown in FIG. 1A of US 2010/0280261, which is shown herein as FIG. 3, is a carrier having a platelet morphology wherein the platelets 130 are approximately the same size and are interlinked to one another. Most platelets 130 appear to intersect and emanate from at least one other platelet. Shown in FIGS. 4-7, 9, 11, 12 and 14 are SEMs of fluorine-mineralized carriers suitable for use herein. FIG. 4 is a SEM of a fluoride-mineralized carrier at 200× magnification. At this magnification, the surface appears to include several circular holes 140 of varying depths. Irregularly shaped valleys 142 and ridges 144 define the surface. FIG. 5 is a SEM at 5000× magnification of another fluoride-mineralized carrier. FIG. 6 is a SEM at 3000× magnification of another fluoride-mineralized carrier. The holes 140 are clearly present. FIG. 7 is a SEM at 5000× magnification of another fluoride-mineralized carrier. FIG. 7 reveals the unexpected presence of both small platelets 170 and large platelets 172 in the same carrier morphology. The carrier shown in FIG. 7 has a variety of platelet sizes which may impact the carrier's properties such as surface area and crush strength. The existence of large plates may result in a carrier with a lower surface area than a carrier wherein essentially all of the platelets are similar in size to small platelets 170. The large platelets may increase the carrier's crush strength by providing greater structural strength. FIG. 9 discloses the morphology of yet another fluoride-mineralized carrier, which at 200× magnification, shows a foam like structure. FIGS. 11 and 12 disclose the morphology of yet another fluoride mineralized carrier at 5000× and 10000×, respectively. In contrast to the carriers shown in FIGS. 4-7, 9, 11 and 12, FIG. 14 discloses a carrier with essentially a monomodal platelet size.

Recognition of the need to control the concentrations of TF, WEF and the ratio of TF:WEF has not been previously recognized in the art of fluoride-mineralized carriers used to manufacture epoxidation catalysts for epoxidation reactions. The concentrations of TF and WEF can be impacted by the choice of raw materials used to manufacture the carriers as well as the conditions of the kiln used to calcine the carriers and post-firing procedures such as rinsing the carrier.

As will be understood by one of skill in the art, the catalytic performance of an ethylene epoxidation catalyst comprising a fluoride-mineralized carrier will generally vary depending upon the particular physical and chemical properties of the fluoride-mineralized carrier used. Accordingly, the ranges disclosed herein with respect to such physical and chemical properties were selected to cover the widest possible variations, the effects of which may be readily determined by experimentation.

3. Ethylene Epoxidation Catalyst Composition

Ethylene epoxidation catalysts suitable for use herein comprise a fluoride-mineralized carrier, as previously described above, and deposited on the fluoride-mineralized carrier, silver and a rhenium promoter. Optionally, an ethylene epoxidation catalyst may further comprise one or more of an alkali metal promoter (e.g., lithium, sodium, potassium, rubidium, cesium, and a combination thereof), a co-promoter (e.g., sulfur, phosphorus, boron, tungsten, molybdenum, chromium, and a combination thereof), a further metal promoter (e.g., alkaline earth metal (such as beryllium, magnesium, calcium, strontium, barium, etc.), titanium, hafnium, zirconium, vanadium, thallium, thorium, tantalum, niobium, gallium, germanium and a combination thereof), and/or a combination thereof.

In broad terms, silver is deposited onto a fluoride-mineralized carrier in an amount sufficient to catalyze the vapor phase reaction of ethylene with oxygen to produce ethylene oxide. When ethylene epoxidation catalysts comprising different amounts of silver are prepared on carriers of similar packing densities, it is convenient to compare the ethylene epoxidation catalysts on a silver weight basis, which is typically expressed in weight percent silver as a function of the total weight of the ethylene epoxidation catalyst. As used herein, unless otherwise specified, the total weight of the ethylene epoxidation catalyst is understood to refer to the weight of the fluoride-mineralized carrier and all components deposited thereon, including silver, rhenium promoter, and any optional promoter(s). Typically, epoxidation catalysts suitable for use herein comprise silver in an amount of 1% to 55% by weight, relative to the total weight of the epoxidation catalyst, or from 1% to 50% by weight, or from 5% to 40% by weight, or from 8% to 35% by weight, or from 10% to 30% by weight, or at least 10% by weight, or at least 15% by weight, or at most 45% by weight, or at most 40% by weight, on the same basis. The upper and lower limits of suitable amounts of silver can be suitably varied, depending upon the particular catalytic performance characteristics or effect desired or the other variables involved, including economic factors.

Alternatively, the amount of silver included in an epoxidation catalyst can be expressed in terms of mass of silver per unit volume of epoxidation catalyst loaded into an epoxidation reactor (e.g., into the catalyst bed). In this way, comparisons of silver loadings between epoxidation catalysts prepared on fluoride-mineralized carriers of different packing densities can be made. Ultimately, the catalyst bed contains a defined volume of epoxidation catalyst, so this method of comparing the amount of silver deposited on an epoxidation catalyst is appropriate. Accordingly, epoxidation catalysts suitable for use herein may comprise silver in an amount of at least 50 kg/m³, relative to the total volume of epoxidation catalyst loaded into the catalyst bed, or at least 100 kg/m³, or at least 125 kg/m³, or at least 150 kg/m³, on the same basis. Similarly, epoxidation catalysts suitable for use herein may comprise silver in an amount of at most 500 kg/m³, relative to the total volume of epoxidation catalyst loaded into the catalyst bed, or at most 450 kg/m³, or at most 400 kg/m³, or at most 350 kg/m³, on the same basis. Preferably, epoxidation catalysts comprise silver in an amount of from 50 to 500 kg/m³, relative to the total volume of epoxidation catalyst loaded into the catalyst bed, or from 100 to 450 kg/m³, or from 125 to 350 kg/m³, on the same basis.

In addition to silver, epoxidation catalysts suitable for use herein further comprise a rhenium promoter and optionally, one or more of an alkali metal promoter, one or more of a co-promoter, one or more of a further metal promoter and/or a combination thereof. Suitable alkali metal promoters may include lithium, sodium, potassium, rubidium, cesium, and a combination thereof. Suitable co-promoters may include sulfur, phosphorus, boron, tungsten, molybdenum, chromium, and a combination thereof. Suitable further metal promoters may include an alkaline earth metal (e.g., beryllium, magnesium, calcium, strontium, barium, etc.), titanium, hafnium, zirconium, vanadium, thallium, thorium, tantalum, niobium, gallium, germanium and a combination thereof. During the reaction to make ethylene oxide, the specific form of the rhenium promoter, alkali metal promoter, co-promoter and further metal promoter may be unknown.

In general, the specific form in which a rhenium promoter or optional promoter(s) is provided is not limited, and may include any of the wide variety of forms known. For example, a rhenium promoter or optional promoter(s) may suitably be provided as an ion (e.g., cation, anion, oxyanion, etc.), or as a compound (e.g., rhenium salts, salts of a co-promoter, alkali metal salts, salts of a further metal promoter, etc.). Generally, suitable compounds are those which can be solubilized in an appropriate solvent, such as a water-containing solvent. As used herein, the term “compound” refers to the combination of a particular element with one or more different elements by surface and/or chemical bonding, such as ionic and/or covalent and/or coordinate bonding. The term “ion” or “ionic” refers to an electrically chemical charged moiety; “cation” or “cationic” being positive, “anion” or “anionic” being negative, and “oxyanion” or “oxyanionic” being a negatively charged moiety containing at least one oxygen atom in combination with another element (i.e., an oxygen-containing anion). It is understood that ions do not exist in vacuo, but are found in combination with charge-balancing counter ions when added. The term “oxidic” refers to a charged or neutral species wherein an element in question is bound to oxygen and possibly one or more different elements by surface and/or chemical bonding, such as ionic and/or covalent and/or coordinate bonding. Thus, an oxidic compound is an oxygen-containing compound which also may be a mixed, double or complex surface oxide. Illustrative oxidic compounds include, but are not limited to, oxides (containing only oxygen as the second element), hydroxides, nitrates, sulfates, carboxylates, carbonates, bicarbonates, oxyhalides, etc. as well as surface species wherein the element in question is bound directly or indirectly to an oxygen either in the substrate or the surface.

As will be appreciated by those of skill in the art, while a specific form of a rhenium promoter or optional promoter(s) may be provided during catalyst preparation, it is possible that during the conditions of preparation of the epoxidation catalyst and/or during use in the epoxidation process, the particular form initially present may be converted to another form. Indeed, once deposited on the fluoride-mineralized carrier and/or during use of the epoxidation catalyst, the specific form of the rhenium promoter or optional promoter(s) is not always known. Furthermore, in many instances, analytical techniques may not be sufficient to precisely identify the form that is present. Accordingly, the present disclosure is not intended to be limited by the exact form of the rhenium promoter and/or optional promoter(s) that may ultimately exist on the ethylene epoxidation catalyst during use. Additionally, it should be understood that while a particular compound may be used during catalyst preparation (e.g., cesium hydroxide is added to an impregnation solution), it is possible that the counter ion added during catalyst preparation may not be present in the finished epoxidation catalyst (e.g., an epoxidation catalyst made using an impregnation solution comprising cesium hydroxide may be analyzed to contain cesium but not hydroxide in the finished epoxidation catalyst).

Ethylene epoxidation catalysts suitable for use herein may comprise a rhenium promoter deposited on a fluoride-mineralized carrier in an amount of 0.01 to 50 mmole/kg, calculated as the amount of rhenium relative to the total weight of the epoxidation catalyst, or from 0.1 to 50 mmole/kg, or from 0.1 to 25 mmole/kg, or from 0.1 to 20 mmole/kg, or from 0.5 to 10 mmole/kg, or from 1 to 6 mmole/kg, or at least 0.01 mmole/kg, or at least 0.1 mmole/kg, or at least 0.5 mmole/kg, or at least 1 mmole/kg, or at least 1.25 mmole/kg, or at least 1.5 mmole/kg, or at most 50 mmole/kg, or at most 20 mmole/kg, or at most 10 mmole/kg, or at most 6 mmole/kg, on the same basis. Alternatively stated, the amount of rhenium promoter, expressed relative to the surface area of the fluoride-mineralized carrier, may preferably be present in the epoxidation catalyst in an amount of from 0.25 to 10 μmole/m², or from 0.5 to 5 μmole/m², or from 1 to 3 μmole/m². For purposes of convenience, the amount of rhenium promoter deposited on the epoxidation catalyst is measured as the metal, irrespective of the form in which it is present.

The degree of benefit obtained within the above-defined concentration limits will vary depending upon one or more properties and characteristics, such as, for example, epoxidation conditions, catalyst preparative conditions, the physical properties and surface chemical properties of the carrier utilized, the amount of silver deposited on the epoxidation catalyst, the amount (if any) of optional promoter(s) deposited, and the amount of other cations and anions present in the epoxidation catalyst, either alone or in combination with the rhenium promoter and/or optional promoter(s). Accordingly, the above-defined limits were selected to cover the widest possible variations in properties and characteristics.

As previously discussed, the specific form in which a rhenium promoter is provided is generally not limited, and may include any of the wide variety of forms known. For example, the rhenium promoter may be provided as the metal, as an ion (e.g., cation, anion, oxyanion, etc.), or as a rhenium compound. Examples of suitable rhenium compounds include, but are not limited to, rhenium salts such as rhenium halides, rhenium oxyhalides, the rhenates, the perrhenates (e.g., ammonium perrhenate, alkali metal perrhenates, alkaline earth metal perrhenates, silver perrhenate, etc.), the oxides and the acids of rhenium. Specific examples of rhenium compounds include, but are not limited to, Re₂O₇, HReO₄, NH₄ReO₄, LiReO₄, NaReO₄, KReO₄, RbReO₄, CsReO₄, and a combination thereof. It should be understood that there are many rhenium compounds that are not soluble per se in water. However, these compounds can be solubilized by utilizing various acids, bases, peroxides, alcohols, etc. After solubilization these compounds could be used, for example, with an appropriate amount of water or other suitable solvent to provide a rhenium promoter. Of course, it is also understood that upon solubilization of many of these compounds, the original compound no longer exists after solubilization. For example, rhenium metal is not soluble in water. However, it is soluble in concentrated nitric acid as well as in hydrogen peroxide solution. Thus, by using an appropriate reactive solvent one could use rhenium metal to provide the rhenium promoter.

Epoxidation catalysts suitable for use herein may further optionally comprise an alkali metal promoter (i.e., lithium, sodium, potassium, rubidium, cesium, or a combination thereof) deposited on a fluoride-mineralized carrier in an amount of 0.01 to 500 mmole/kg, calculated as the amount of the element relative to the total weight of the epoxidation catalyst, or from 0.01 to 400 mmole/kg, or from 0.1 to 300 mmole/kg, or from 0.1 to 250 mmole/kg, or from 0.5 to 200 mmole/kg, or from 1 to 100 mmole/kg, or at least 0.01 mmole/kg, or at least 0.05, or at least 0.1 mmole/kg, or at least 0.5 mmole/kg, or at least 1 mmole/kg, or at least 1.25 mmole/kg, or at least 1.5 mmole/kg, or at least 2 mmole/kg, or at least 3 mmole/kg, or at most 500 mmole/kg, or at most 400 mmole/kg, or at most 300 mmole/kg, or at most 250 mmole/kg, or at most 200 mmole/kg, or at most 150 mmole/kg, or at most 100 mmole/kg, on the same basis. For purposes of convenience, the amount of the alkali metal deposited on the epoxidation catalyst is measured as the element, irrespective of the form in which it is present.

It should be understood that the amount of an alkali metal promoter deposited on the fluoride-mineralized carrier is not necessarily the total amount of alkali metal present in the epoxidation catalyst. Rather, the amount deposited reflects the amount of alkali metal promoter that has been added to the fluoride-mineralized carrier (e.g., via impregnation). As such, the amount of alkali metal promoter deposited on the fluoride-mineralized carrier does not include any amount of alkali metals that may be locked into the carrier, for example, by calcining, or are not extractable in a suitable solvent such as water or lower alkanol or amine or mixtures thereof and do not provide a promoting effect. It is also understood that the source of the alkali metal promoter may be the fluoride-mineralized carrier itself. That is, the fluoride-mineralized carrier may contain extractable amounts of an alkali metal promoter that can be extracted with a suitable solvent, such as water or lower alkanol, thus preparing a solution from which the alkali metal promoter may be deposited or redeposited on the fluoride-mineralized carrier.

The degree of benefit obtained within the above-defined concentration limits will vary depending upon one or more properties and characteristics, such as, for example, epoxidation conditions, catalyst preparative conditions, the physical properties and surface chemical properties of the carrier utilized, the amount of silver deposited on the epoxidation catalyst, the amount of rhenium promoter deposited on the epoxidation catalyst, the amount (if any) of a co-promoter and/or further metal promoter deposited on the epoxidation catalyst, and the amount of other cations and anions present in the epoxidation catalyst, either alone or in combination with the rhenium promoter and/or optional promoter(s). Accordingly, the above-defined limits were selected to cover the widest possible variations in properties and characteristics.

As previously discussed, the specific form in which an alkali metal promoter is provided is generally not limited, and may include any of the wide variety of forms known. For example, the alkali metal promoter may be provided as an ion (e.g., cation), or as an alkali metal compound. Examples of suitable alkali metal compounds include, but are not limited to, alkali metal salts and oxidic compounds of the alkali metals, such as the nitrates, nitrites, carbonates, bicarbonates, oxalates, carboxylic acid salts, hydroxides, halides, oxyhalides, borates, sulfates, sulfites, bisulfates, acetates, tartrates, lactates, oxides, peroxides, and iso-propoxides, etc.

As previously mentioned, the alkali metal promoter may comprise a combination of two or more alkali metal promoters. Non-limiting examples include a combination of cesium and rubidium, a combination of cesium and potassium, a combination of cesium and sodium, a combination of cesium and lithium, a combination of cesium, rubidium and sodium, a combination of cesium, potassium and sodium, a combination of cesium, lithium and sodium, a combination of cesium, rubidium and sodium, a combination of cesium, rubidium, potassium and lithium, and a combination of cesium, potassium, and lithium.

Furthermore, in those embodiments where an epoxidation catalyst comprises a combination of two or more alkali metal promoters, it may be particularly beneficial if the alkali metal promoters comprise potassium and at least one additional alkali metal promoter selected from cesium, rubidium, and a combination thereof, preferably cesium. The amount of potassium deposited on the fluoride-mineralized carrier may be in an amount of 0.01 to 50 mmole/kg, calculated as the amount of the element relative to the total weight of the epoxidation catalyst, or from 0.1 to 400 mmole/kg, or from 0.2 to 30 mmole/kg, or from 0.5 to 20 mmole/kg, or from 1 to 15 mmole/kg, or from 1.5 to 10 mmole/kg, or from 2 to 8 mmole/kg, or at least 0.01 mmole/kg, or at least 0.1 mmole/kg, or at least 0.2, or at least 0.5 mmole/kg, or at least 1 mmole/kg, or at least 1.25 mmole/kg, or at least 1.5 mmole/kg, or at least 1.75 mmole/kg, or at least 2 mmole/kg, or at least 3 mmole/kg, or at most 40 mmole/kg, or at most 35 mmole/kg, or at most 30 mmole/kg, or at most 25 mmole/kg, or at most 20 mmole/kg, or at most 15 mmole/kg, or at most 10 mmole/kg, on the same basis. The amount of the at least one additional alkali metal promoter selected from cesium, rubidium, and a combination thereof deposited on the fluoride-mineralized carrier may be in an amount of 0.1 to 40 mmole/kg, calculated as the amount of the element (e.g., cesium and/or rubidium) relative to the total weight of the epoxidation catalyst, or from 0.2 to 35 mmole/kg, or from 0.25 to 30 mmole/kg, or from 0.5 to 20 mmole/kg, or from 1 to 15 mmole/kg, or from 3 to 10 mmole/kg, or at least 0.1 mmole/kg, or at least 0.15, or at least 0.2 mmole/kg, or at least 0.25 mmole/kg, or at least 0.3 mmole/kg, or at least 0.35 mmole/kg, or at least 0.4 mmole/kg, or at least 0.45 mmole/kg, or at least 0.5 mmole/kg, or at most 40 mmole/kg, or at most 35 mmole/kg, or at most 30 mmole/kg, or at most 25 mmole/kg, or at most 20 mmole/kg, or at most 15 mmole/kg, or at most 10 mmole/kg, on the same basis. Further, it may be beneficial to deposit the potassium and the at least one additional alkali metal promoter selected from cesium, rubidium, and a combination thereof in an amount such that the molar ratio of potassium to the additional alkali metal promoter is at least 0.25, or at least 0.5, at least 0.75, at least 1, or at least 1.25, or at most 20, at most 15, at most 10, or at most 7.5, or at most 5.

Further, in those embodiments where the alkali metal promoter comprises a combination of potassium and at least one additional alkali metal promoter selected from cesium, rubidium, and a combination thereof, it may be additionally advantageous to deposit a third alkali metal promoter selected from the group consisting of lithium, sodium and a combination thereof, preferably lithium. The amount of the third alkali metal promoter selected from lithium, sodium and a combination thereof deposited on the fluoride-mineralized carrier may be in an amount of 0.1 to 400 mmole/kg, calculated as the amount of the element (e.g., lithium and/or sodium) relative to the total weight of the epoxidation catalyst, or from 0.5 to 350 mmole/kg, or from 1 to 300 mmole/kg, or from 1 to 200 mmole/kg, or from 1 to 150 mmole/kg, or from 5 to 100 mmole/kg, or at least 0.1 mmole/kg, or at least 0.1, or at least 0.25 mmole/kg, or at least 0.5 mmole/kg, or at least 0.75 mmole/kg, or at least 1 mmole/kg, or at least 2.5 mmole/kg, or at least 5 mmole/kg, or at most 400 mmole/kg, or at most 350 mmole/kg, or at most 300 mmole/kg, or at most 250 mmole/kg, or at most 200 mmole/kg, or at most 150 mmole/kg, or at most 100 mmole/kg, on the same basis.

Further, in those embodiments where the alkali metal promoter comprises potassium, it may be particularly advantageous if the fluoride-mineralized carrier contains nitric acid leachable potassium in a quantity of less than 85 parts per million by weight (“ppmw”), relative to the weight of the fluoride mineralized carrier, or less than 80 ppmw, less than 75 ppmw, or less than 65 ppmw, on the same basis. The quantity of nitric acid leachable potassium is deemed to be the quantity insofar as it can be extracted from the fluoride-mineralized carrier. The extraction involves extracting a 10-gram sample of the fluoride-mineralized carrier with 100 mL of 10% w nitric acid for 30 minutes at 100° C. (1 atm) and determining the amount of potassium present in the extract using standard Atomic Absorption spectroscopy techniques. Similarly, in those embodiments where the alkali metal promoter comprises potassium, it may also be advantageous if the fluoride-mineralized carrier contains water leachable potassium in a quantity of less than 40 ppmw, relative to the weight of the fluoride-mineralized carrier, less than 35 ppmw, or less than 30 ppmw, on the same basis. The quantity of water leachable potassium in the fluoride-mineralized carrier is deemed to be the quantity insofar as it can be extracted from the fluoride-mineralized carrier. The extraction involves extracting a 2-gram sample of the fluoride-mineralized carrier three times by heating it in 25-gram portions of de-ionized water for 5 minutes at 100° C. and determining in the combined extracts the amount of alkali metal by using a known method, for example atomic absorption spectroscopy.

In these embodiments, potassium may be deposited in a quantity of at least 0.5 mmole/kg, at least 1 mmole/kg, at least 1.5 mmole/kg, at least 1.75 mmole/kg, calculated as the total quantity of the potassium deposited relative to the weight of the catalyst. Similarly, potassium may be deposited in an amount of at most 20 mmole/kg, at most 15 mmole/kg, at most 10 mmole/kg, at most 5 mmole/kg, on the same basis. Potassium may be deposited in an amount in the range of from 0.5 to 20 mmole/kg, from 1 to 15 mmole/kg, from 1.5 to 7.5 mmole/kg, from 1.75 to 5 mmole/kg, on the same basis. Additionally, it may be advantageous, if the epoxidation catalyst comprises potassium in an amount such that the amount of water extractable potassium of the catalyst may be at least 1.25 mmole/kg, relative to the weight of the epoxidation catalyst, at least 1.5 mmole/kg, or at least 1.75 mmole/kg, on the same basis. Suitably, the epoxidation catalyst may comprise water extractable potassium in an amount of at most 10 mmole/kg, at most 7.5 mmole/kg, at most 5 mmole/kg, on the same basis. Suitably, the epoxidation catalyst may comprise water extractable potassium in an amount in the range of from 1.25 to 10 mmole/kg, from 1.5 to 7.5 mmole/kg, or from 1.75 to 5 mmole/kg, on the same basis. The source of water extractable potassium may originate from the fluoride-mineralized carrier and/or the components of the epoxidation catalyst. The quantity of water extractable potassium in the catalyst is deemed to be the quantity insofar as it can be extracted from the catalyst. The extraction involves extracting a 2-gram sample of the catalyst three times by heating it in 25-gram portions of de-ionized water for 5 minutes at 100° C. and determining in the combined extracts the amount of potassium by using a known method, for example atomic absorption spectroscopy.

Optionally, epoxidation catalysts suitable for use herein may further comprise a co-promoter (e.g., sulfur, phosphorus, boron, tungsten, molybdenum, chromium, or a combination thereof) deposited on a fluoride-mineralized carrier in an amount of 0.01 to 500 mmole/kg, calculated as the amount of the element relative to the total weight of the epoxidation catalyst, or from 0.01 to 100 mmole/kg, or from 0.1 to 50 mmole/kg, or from 0.1 to 20 mmole/kg, or from 0.5 to 10 mmole/kg, or from 1 to 6 mmole/kg, or at least 0.01 mmole/kg, or at least 0.05, or at least 0.1 mmole/kg, or at least 0.5 mmole/kg, or at least 1 mmole/kg, or at least 1.25 mmole/kg, or at least 1.5 mmole/kg, or at least 2 mmole/kg, or at least 3 mmole/kg, or at most 100 mmole/kg, or at most 50 mmole/kg, or at most 40 mmole/kg, or at most 30 mmole/kg, or at most 20 mmole/kg, or at most 10 mmole/kg, or at most 5 mmole/kg, on the same basis. For purposes of convenience, the amount of co-promoter deposited on the epoxidation catalyst is measured as the element, irrespective of the form in which it is present.

The degree of benefit obtained within the above-defined concentration limits will vary depending upon one or more properties and characteristics, such as, for example, epoxidation conditions, catalyst preparative conditions, the physical properties and surface chemical properties of the carrier utilized, the amount of silver deposited on the epoxidation catalyst, the amount of rhenium promoter and alkali metal promoter deposited on the epoxidation catalyst, the amount (if any) of further metal promoter deposited on the epoxidation catalyst, and the amount of other cations and anions present in the epoxidation catalyst, either alone or in combination with the rhenium promoter, co-promoter, alkali metal promoter and/or further metal promoter. Accordingly, the above-defined limits were selected to cover the widest possible variations in properties and characteristics.

As previously discussed, the specific form in which a co-promoter is provided is generally not limited, and may include any of the wide variety of forms known. For example, the co-promoter may be provided as an ion (e.g., cation, anion, oxyanion, etc.), or as a co-promoter compound (e.g., salts of the co-promoters). Examples of suitable co-promoter compounds include, but are not limited to, salts of the co-promoter elements, such as the oxyanionic compounds of the co-promoter elements (e.g., ammonium oxyanionates, such ammonium sulfate, ammonium molybdate, etc.; alkali metal oxyanionates, such as potassium sulfate, cesium chromate, rubidium tungstate, lithium sulfate, sodium tungstate, lithium chromate, etc.). Specific examples of anions of sulfur that can be suitably applied include sulfate, sulfite, bisulfite, bisulfate, sulfonate, persulfate, thiosulfate, dithionate, dithionite, etc. Specific examples of anions of phosphorus and boron that can be suitably applied include phosphate, polyphosphates, etc.; and borates, etc. Specific examples of anions of molybdenum, tungsten and chromium that can be suitably applied include molybdate, dimolybdate, paramolybdate, other iso- and hetero-polymolybdates, etc.; tungstate, paratungstate, metatungstate, other iso- and hetero-polytungstates, etc.; and chromate, dichromate, chromite, halochromate, etc. The anions can be supplied with various counter-ions (e.g., ammonium, alkali metal, alkaline earth metal, and hydrogen (i.e., acid form)). The anions can be prepared by the reactive dissolution of various non-anionic materials, such as the oxides (e.g., SO₂, SO₃, MoO₃, WO₃, Cr₂O₃, etc.), as well as other materials such as halides, oxyhalides, hydroxyhalides, hydroxides, sulfides, etc., of the co-promoter elements.

In those embodiments where an epoxidation catalyst comprises a co-promoter, it may be particularly beneficial if the co-promoter comprises a combination of a first co-promoter selected from sulfur, phosphorus, boron, and a combination thereof, and a second co-promoter selected from the group consisting of tungsten, molybdenum, chromium, and a combination thereof. The amount of the first co-promoter deposited on the fluoride-mineralized carrier may be in an amount of 0.2 to 50 mmole/kg, calculated as the amount of the element (e.g., sulfur, phosphorus and/or boron) relative to the total weight of the epoxidation catalyst, or from 0.5 to 45 mmole/kg, or from 0.5 to 30 mmole/kg, or from 1 to 20 mmole/kg, or from 1.5 to 10 mmole/kg, or from 2 to 6 mmole/kg, or at least 0.2 mmole/kg, or at least 0.3, or at least 0.5 mmole/kg, or at least 1 mmole/kg, or at least 1.25 mmole/kg, or at least 1.5 mmole/kg, or at least 1.75 mmole/kg, or at least 2 mmole/kg, or at least 3 mmole/kg, or at most 50 mmole/kg, or at most 45 mmole/kg, or at most 40 mmole/kg, or at most 35 mmole/kg, or at most 30 mmole/kg, or at most 20 mmole/kg, or at most 10 mmole/kg, or at most 6 mmole/kg, on the same basis. The amount of the second co-promoter deposited on the fluoride-mineralized carrier may be in an amount of 0.1 to 40 mmole/kg, calculated as the amount of the element (e.g., tungsten, molybdenum and/or chromium) relative to the total weight of the epoxidation catalyst, or from 0.15 to 30 mmole/kg, or from 0.2 to 25 mmole/kg, or from 0.25 to 20 mmole/kg, or from 0.3 to 10 mmole/kg, or from 0.4 mmole/kg to 5 mmole/kg, or at least 0.1 mmole/kg, or at least 0.15, or at least 0.2 mmole/kg, or at least 0.25 mmole/kg, or at least 0.3 mmole/kg, or at least 0.35 mmole/kg, or at least 0.4 mmole/kg, or at least 0.45 mmole/kg, or at least 0.5 mmole/kg, or at most 40 mmole/kg, or at most 35 mmole/kg, or at most 30 mmole/kg, or at most 25 mmole/kg, or at most 20 mmole/kg, or at most 15 mmole/kg, or at most 10 mmole/kg, or at most 5 mmole/kg, on the same basis. Further, it may be beneficial to deposit the first and second co-promoters in an amount such that the molar ratio of the first co-promoter to the second co-promoter is greater than 1, or at least 1.25, at least 1.5, at least 2, or at least 2.5. It is further preferred that the molar ratio of the first co-promoter to the second co-promoter is at most 20, at most 15, at most 10, or at most 7.5. Additionally, it is preferred that the molar ratio of the rhenium promoter to the second co-promoter may be greater than 1, at least 1.25, or at least 1.5. It is further preferred that the molar ratio of the rhenium promoter to the second co-promoter may be at most 20, at most 15, or at most 10.

Optionally, epoxidation catalysts suitable for use herein may additionally comprise a further metal promoter (e.g., an alkaline earth metal such as beryllium, magnesium, calcium, strontium, barium, etc., titanium, hafnium, zirconium, vanadium, thallium, thorium, tantalum, niobium, gallium, germanium, manganese, etc.) deposited on a fluoride-mineralized carrier in an amount of 0.01 to 500 mmole/kg, calculated as the amount of the element relative to the total weight of the epoxidation catalyst, or from 0.01 to 100 mmole/kg, or from 0.1 to 50 mmole/kg, or from 0.1 to 20 mmole/kg, or from 0.5 to 10 mmole/kg, or from 1 to 6 mmole/kg, or at least 0.01 mmole/kg, or at least 0.05, or at least 0.1 mmole/kg, or at least 0.5 mmole/kg, or at least 1 mmole/kg, or at least 1.25 mmole/kg, or at least 1.5 mmole/kg, or at least 2 mmole/kg, or at least 3 mmole/kg, or at most 100 mmole/kg, or at most 50 mmole/kg, or at most 40 mmole/kg, or at most 30 mmole/kg, or at most 20 mmole/kg, or at most 10 mmole/kg, or at most 5 mmole/kg, on the same basis. For purposes of convenience, the amount of further metal promoter in the epoxidation catalyst is measured as the element, irrespective of the form in which it is present.

The degree of benefit obtained within the above-defined concentration limits will vary depending upon one or more properties and characteristics, such as, for example, epoxidation conditions, catalyst preparative conditions, the physical properties and surface chemical properties of the carrier utilized, the amount of silver deposited on the epoxidation catalyst, the amount of rhenium promoter and alkali metal promoter deposited on the epoxidation catalyst, the amount (if any) of co-promoter deposited on the epoxidation catalyst, and the amount of other cations and anions present in the epoxidation catalyst, either alone or in combination with the rhenium promoter, alkali metal promoter and/or co-promoter. Accordingly, the above-defined limits were selected to cover the widest possible variations in properties and characteristics.

As previously discussed, the specific form in which a further metal promoter is provided is generally not limited, and may include any of the wide variety of forms known. For example, the further metal promoter may be provided as an ion (e.g., cation, anion, oxyanion, etc.), or as a compound (e.g., salts of the further metals). Examples of suitable compounds include, but are not limited to, salts of the further metals, such as alkaline earth metal salts (e.g., the nitrates, nitrites, carbonates, bicarbonates, oxalates, carboxylic acid salts, hydroxides, halides, oxyhalides, borates, sulfates, sulfites, bisulfates, acetates, tartrates, lactates and iso-propoxides, etc.), and the oxides, halides and oxyhalides of the further metals.

Well known methods can be employed to analyze for the amounts of silver, rhenium promoter and optional promoter(s) deposited onto the fluoride-mineralized carrier. The skilled artisan may employ, for example, material balances to determine the amounts of any of these deposited components. As an example, if the fluoride-mineralized carrier is weighed prior to and after deposition of silver and a rhenium promoter, then the difference in the two weights will be equal to the amount of silver and the rhenium promoter deposited onto the fluoride-mineralized carrier, from which the amount of the deposited rhenium promoter can be calculated. Additionally, the amount of the deposited silver and promoters can be calculated based upon the ratio of the concentration of silver and promoters included in the impregnation solution(s) and the total weight in the finished epoxidation catalyst.

Alternatively, the amount of promoters deposited on the fluoride-mineralized carrier may also be determined by known leaching methods, wherein the amount of metallic leachables present in the fluoride-mineralized carrier and the amount of metallic leachables present in the epoxidation catalyst are independently determined and the difference between the two measurements reflect the total amount of promoter deposited on the fluoride-mineralized carrier. As an example, the amount of an alkali metal promoter deposited on an epoxidation catalyst may be determined by separately leaching a 10-gram sample of the fluoride-mineralized carrier and a 10-gram sample of the epoxidation catalyst with 100 mL of 10% w nitric acid for 30 minutes at 100° C. (1 atm) and determining the amount of the alkali metal promoter present in the extracts using standard Atomic Absorption spectroscopy techniques. The difference in the measurements between the carrier and the catalyst reflect the amount of alkali metal promoter deposited onto the carrier.

4. Epoxidation Catalyst Preparation

The preparation of epoxidation catalysts comprising silver is known in the art. The specific manner in which epoxidation catalysts suitable for use herein are prepared is not limited, and therefore any method known in the art may be used. Reference is made to U.S. Pat. Nos. 4,761,394, 4,766,105, 5,380,697, 5,739,075, 6,368,998 and 6,656,874, which are incorporated herein by reference, for descriptions relating to the preparation of epoxidation catalysts.

In general, an epoxidation catalyst suitable for use herein is prepared by contacting (e.g., impregnating) a fluoride-mineralized carrier with one or more solutions comprising silver, a rhenium promoter and, if desired, optional promoter(s); and subsequently depositing silver, the rhenium promoter and, if desired, any optional promoter(s), on the fluoride-mineralized carrier, typically by heating the impregnated carrier.

As used herein, the phrase “contacting a fluoride-mineralized carrier with one or more solutions comprising silver, a rhenium promoter and, if desired, optional promoter(s)” and similar or cognate terminology means that the fluoride-mineralized carrier is contacted (e.g., impregnated) in a single or multiple step with one solution comprising silver, a rhenium promoter and, if desired, optional promoter(s); or in multiple steps with two or more solutions, wherein each solution comprises at least one component selected from silver, a rhenium promoter and, if desired, optional promoter(s), with the proviso that all of the components of silver, a rhenium promoter and if desired, optional promoter(s), will individually be found in at least one of the solutions. Furthermore, as is known in the art, the sequence of contacting the fluoride-mineralized carrier with one or more solutions comprising silver, a rhenium promoter and, if desired, optional promoter(s), as well as the sequence of depositing these components on the fluoride-mineralized carrier, may vary. Thus, impregnation and deposition of silver, a rhenium promoter and if desired, optional promoter(s), may be effected coincidentally or sequentially. For example, a rhenium promoter and, if desired, optional promoter(s) may be deposited on a fluoride-mineralized carrier either prior to, simultaneously with, or subsequent to the deposition of silver and each other. Similarly, the rhenium promoter and optional promoter(s) may be deposited together or sequentially. Furthermore, for example, silver may be deposited first followed by the coincidental or sequential deposition of a rhenium promoter and if desired, optional promoter(s); or alternatively, a rhenium promoter may be deposited first followed by coincidental or sequential deposition of silver and if desired, any optional promoter(s); or alternatively, an optional promoter may be deposited first followed by coincidental or sequential deposition of silver and a rhenium promoter. If two or more impregnations are employed, the impregnated carrier is typically dried, or heated between each successive impregnation to ensure deposition of the components onto the carrier. Furthermore, if it is desired for the epoxidation catalyst to comprise silver in an amount greater than 25% by weight, it is often necessary to subject the fluoride-mineralized carrier to at least two or more sequential impregnations of a solution comprising silver to obtain the desired amount of silver deposited on the carrier.

Although epoxidation catalysts suitable for use herein are typically prepared by impregnating a fluoride-mineralized carrier with one or more solutions (commonly referred to as “impregnation solution(s)”) comprising silver, a rhenium promoter and, if desired, optional promoter(s), the present disclosure is not intended to be limited to any particular preparation method. Accordingly, any known preparative method may be used provided that the silver, rhenium promoter and optional promoter(s) (if any) are deposited on the fluoride-mineralized carrier in a suitable manner. For example, alternatively, a coating of silver, rhenium promoter and if desired, optional promoter(s), may be formed on a fluoride-mineralized carrier from one or more emulsions or slurries containing the components.

With regards to the specific form of silver used in the one or more solutions, any of the wide variety of forms known may be used, provided that the silver can be solubilized therein. For example, silver may suitably be provided as a silver compound, such as, a silver complex or a silver salt, such as silver nitrate, silver oxide, silver carbonate, and silver salts of mono- and polybasic carboxylic and hydroxycarboxylic acids of up to 16 carbon atoms, such as silver acetate, propionate, butyrate, oxalate, malonate, malate, maleate, lactate, citrate, phthalate, higher fatty acids salts, combinations thereof and the like. Likewise, as previously mentioned, the specific form in which the rhenium promoter and optional promoter(s) (if any) is provided is not critical, provided that they can be solubilized in an appropriate solvent and do not undesirably react with other components present in the solution. For example, when an alkali metal promoter is coincidentally deposited with silver, the alkali metal promoter employed is preferably one which does not react with the silver compound (e.g., silver salt) in solution in order to avoid premature silver precipitation from the same.

A wide variety of solvents or complexing/solubilizing agents may be employed in the one or more solutions to solubilize silver, the rhenium promoter and/or any optional promoter(s) to the desired concentration in the solution. The solvent used is not particularly limited and may include any solvent or agent capable of adequately dissolving the silver compound or converting the silver compound to a soluble form, or if the solution comprises a rhenium promoter and/or optional promoter(s), it should be capable of adequately dissolving or converting these components to a soluble form. Furthermore, suitable solvents or complexing/solubilizing agents should be capable of being readily removed in subsequent steps, either by a washing, volatilizing or oxidation procedure, or the like. Preferably, the solvent or complexing/solubilizing agent is readily miscible with water, as aqueous solutions may conveniently be employed. Examples of suitable solvents or complexing/solubilizing agents include, but are not limited to, alcohols, including glycols, such as ethylene glycol, ammonia, amines and aqueous mixtures of amines, carboxylic acids, such as lactic acid, and mixtures thereof. Additionally, examples of suitable amines include, but are not limited to, organic amines, such as, lower alkylenediamines of from 1 to 5 carbon atoms (e.g., ethylenediamine), mixtures of a lower alkanolamine of from 1 to 5 carbon atoms with a lower alkylenediamine of from 1 to 5 carbon atoms (e.g., ethylenediamine in combination with ethanolamine), as well as mixtures of ammonia with lower alkanolamines or lower alkylenediamines of from 1 to 5 carbons (e.g., ethanolamine in combination with ammonia, ethylenediamine in combination with ammonia). In those solutions comprising silver, these solubilizing/reducing agents are generally added in the amount of from 0.1 to 10 moles per mole of silver present.

Optionally, the one or more solutions may further comprise a base, such as a metal hydroxide (e.g., lithium hydroxide, cesium hydroxide, rubidium hydroxide, sodium hydroxide), an alkylammonium hydroxide (e.g., tetraalkylammonium hydroxides, such as tetramethylammonium hydroxide or tetraethylammonium hydroxide), 1,8-bis-(dimethylamino)-naphthalene, or a combination thereof, in an amount sufficient to provide a solution having a pH of above 11.2, more typically at least 11.7, preferably at least 12, as measured at 20° C. It should be understood that the pH of the solution may not be a true pH when the solution is not aqueous. Furthermore, if a base is included, it is often desirable to select a base that does not alter the metal concentration of the one or more solutions, such as an organic base; however, if changing the metals concentration of the solution is not a concern, metal bases may be used.

Following impregnation of the fluoride-mineralized carrier with the one or more solutions, the carrier is typically separated from any remaining non-absorbed solution (e.g., by draining the excess solution, or by using separation techniques, such as filtration, centrifugation or evaporation under reduced pressure at a suitable temperature) and the silver, the rhenium promoter and, if desired, any optional promoter(s) are deposited on the carrier, most often by heating (also referred to as “roasting”). In general, the impregnated carrier is heated at a temperature that is high enough, and for a period of time that is sufficiently long enough, to cause reduction of the silver compound (e.g., silver complex) to metallic silver and to form a layer of finely divided silver, which is bound to the surface of the fluoride-mineralized carrier, both the exterior and pore surface. It is observed that independent of the form in which the silver is present in the solution before precipitation on the fluoride-mineralized carrier, the phrase “reduction of the silver compound to metallic silver” is used, while in the meantime often decomposition of the silver compound by heating occurs. The term “reduction” is preferably used herein in view of the conversion of the positively charged Ag⁺ ion into metallic Ag atom.

Generally, an impregnated carrier may be heated at a temperature of from 100° C. to 600° C. for a period of time ranging from 0.01 to 12 hours. The pressure during heating is preferably atmospheric pressure. As would be recognized by one skilled in the art, if heating is conducted at a lower temperature, a longer period of time is generally required and likewise, if heating is conducted at a higher temperature, less time is typically required. Although it is provided herein that heating should generally be conducted at a temperature in a range of from 100° C. to 600° C., for a period of time from 0.01 to 12 hours, and at atmospheric pressure, the present disclosure is nevertheless independent of the manner by which such heating is conducted. Thus, variations in heating known in the art, such as holding at one temperature for a certain period of time and then raising the temperature to a second temperature over the course of a second period of time, are contemplated by the present disclosure. Furthermore, heating may be carried out in any suitable atmosphere, such as air, or other oxidizing gas, reducing gas, inert gas or mixtures thereof. The equipment used for such heating may use a static or flowing atmosphere of such gases to effect reduction, preferably a flowing atmosphere.

Optionally, the impregnated carrier may be dried in the presence of an atmosphere which reduces the silver compound to metallic silver. Drying methods known in the art include steam drying, drying in an atmosphere with a controlled oxygen concentration, drying in a reducing atmosphere, and air drying.

After reduction, suitable silver particle sizes may be in the range of from 10 to 10,000 angstroms in diameter, or from greater than 100 to less than 5,000 angstroms in diameter. Although not necessary, it is generally preferred for the silver to be relatively uniformly deposited on the fluoride-mineralized carrier.

Therefore, the present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered or modified and all such variations are considered within the scope and spirit of the present invention. While compositions and methods/processes are described in terms of “comprising,” “containing,” or “including” various components or steps, the compositions and methods/processes can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from a to b,” or, equivalently, “from a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the element that it introduces. If there is any conflict in the usages of a word or term in this specification and one or more patent or other documents that may be incorporated herein by reference, the definitions that are consistent with this specification should be adopted.

What is claimed is:

1. An ethylene epoxidation catalyst comprising a fluoride-mineralized carrier having silver and a rhenium promoter deposited thereon, wherein the fluoride-mineralized carrier has:
a total fluorine (TF) content less than 5000 ppm as measured by XRF,
a water extractable fluorine (WEF) content greater than 45 ppm as measured by microwave extraction and ion specific electrode, and
wherein the ratio of TF:WEF is between 10 and 110.

2. The ethylene epoxidation catalyst of claim 1 wherein the ratio of TF:WEF is greater than 15.

3. The ethylene epoxidation catalyst of claim 1 wherein the ratio of TF:WEF is less than 90.

4. The ethylene epoxidation catalyst of claim 1 wherein the ratio of TF:WEF is less than 70.

5. The ethylene epoxidation catalyst of claim 1 wherein the WEF is greater than 50 ppm.

6. The ethylene epoxidation catalyst of claim 1 wherein the WEF is greater than 100 ppm.

7. The ethylene epoxidation catalyst of claim 1 wherein the WEF is less than 300 ppm.

8. The ethylene epoxidation catalyst of claim 1 wherein the WEF is less than 200 ppm.

9. The ethylene epoxidation catalyst of claim 1 wherein the WEF is less than 180 ppm.

10. The ethylene epoxidation catalyst of claim 1 wherein the TF is greater than 2000 ppm.

11. The ethylene epoxidation catalyst of claim 1 wherein the TF is greater than 2500 ppm.

12. The ethylene epoxidation catalyst of claim 1 wherein the TF is less than 4500 ppm.

13. The ethylene epoxidation catalyst of claim 1 wherein the fluoride-mineralized carrier has a water absorption of at least 50 weight percent.

14. The ethylene epoxidation catalyst of claim 1 wherein the fluoride-mineralized carrier has a silica content less than 0.05 weight percent.

15. The ethylene epoxidation catalyst of claim 1 wherein the fluoride-mineralized carrier has a magnesia content less than 0.05 weight percent.

16. The ethylene epoxidation catalyst of claim 1 wherein the fluoride-mineralized carrier has a surface area of from 0.5 m²/g to 5 m²/g.

17. The ethylene epoxidation catalyst of claim 1 wherein the fluoride-mineralized carrier is multi-lobed.

18. The ethylene epoxidation catalyst of claim 1 further comprising an alkali metal promoter selected from the group consisting of lithium, sodium, potassium, rubidium, cesium, and a combination thereof.

19. The ethylene epoxidation catalyst of claim 18 further comprising:
a first co-promoter selected from the group consisting of sulfur, phosphorus, boron, and a combination thereof; and
a second co-promoter selected from the group consisting of tungsten, molybdenum, chromium, and a combination thereof.

20. The ethylene epoxidation catalyst of claim 19 further comprising a further metal selected from the group consisting of beryllium, magnesium, calcium, strontium, barium, titanium, hafnium, zirconium, vanadium, thallium, thorium, tantalum, niobium, gallium, germanium, manganese, and a combination thereof.

21. A method for the epoxidation of ethylene comprising:
contacting an inlet feed gas comprising ethylene and oxygen with an ethylene epoxidation catalyst comprising a fluoride-mineralized carrier having silver and a rhenium promoter deposited thereon, wherein the fluoride-mineralized carrier has:
a total fluorine (TF) content less than 5000 ppm as measured by XRF,
a water extractable fluorine (WEF) content greater than 45 ppm as measured by microwave extraction and ion specific electrode, and
wherein the ratio of TF:WEF is between 10 and 110.

22. The method of claim 21 wherein the ratio of TF:WEF is greater than 15.

23. The method of claim 21 wherein the ratio of TF:WEF is less than 90.

24. The method of claim 21 wherein the ratio of TF:WEF is less than 70.

25. The method of claim 21 wherein the WEF is greater than 50 ppm.

26. The method of claim 21 wherein the WEF is greater than 100 ppm.

27. The method of claim 21 wherein the WEF is less than 300 ppm.

28. The method of claim 21 wherein the WEF is less than 200 ppm.

29. The method of claim 21 wherein the WEF is less than 180 ppm.

30. The method of claim 21 wherein the TF is greater than 2000 ppm.

31. The method of claim 21 wherein the TF is greater than 2500 ppm.

32. The method of claim 21 wherein the TF is less than 4500 ppm.

33. The method of claim 21 wherein the fluoride-mineralized carrier has a water absorption of at least 50 weight percent.

34. The method of claim 21 wherein the fluoride-mineralized carrier has a silica content less than 0.05 weight percent.

35. The method of claim 21 wherein the fluoride-mineralized carrier has a magnesia content less than 0.05 weight percent.

36. The method of claim 21 wherein the fluoride-mineralized carrier has a surface area of from 0.5 m²/g to 5 m²/g.

37. The method of claim 21 wherein the fluoride-mineralized carrier has a surface area of from 0.7 m²/g to 3 m²/g.

38. The method of claim 21 wherein the fluoride-mineralized carrier is multi-lobed.

39. The method of claim 21 wherein the ethylene epoxidation catalyst further comprises an alkali metal promoter selected from the group consisting of lithium, sodium, potassium, rubidium, cesium, and a combination thereof.

40. The method of claim 39 wherein the ethylene epoxidation catalyst further comprises:
a first co-promoter selected from the group consisting of sulfur, phosphorus, boron, and a combination thereof; and
a second co-promoter selected from the group consisting of tungsten, molybdenum, chromium, and a combination thereof.

41. The method of claim 40 wherein the ethylene epoxidation catalyst further comprises a further metal selected from the group consisting of beryllium, magnesium, calcium, strontium, barium, titanium, hafnium, zirconium, vanadium, thallium, thorium, tantalum, niobium, gallium, germanium, manganese, and a combination thereof.

42. A method for manufacturing an ethylene epoxidation catalyst comprising:
depositing silver and a rhenium promoter on a fluoride-mineralized carrier, wherein the fluoride-mineralized carrier has:
a total fluorine (TF) content less than 5000 ppm as measured by XRF,
a water extractable fluorine (WEF) content greater than 45 ppm as measured by microwave extraction and ion specific electrode, and
wherein the ratio of TF:WEF is between 10 and 110.

43. The method of claim 42 wherein the ratio of TF:WEF is greater than 15.

44. The method of claim 42 wherein the ratio of TF:WEF is less than 90.

45. The method of claim 42 wherein the ratio of TF:WEF is less than 70.

46. The method of claim 42 wherein the WEF is greater than 50 ppm.

47. The method of claim 42 wherein the WEF is greater than 100 ppm.

48. The method of claim 42 wherein the WEF is less than 300 ppm.

49. The method of claim 42 wherein the WEF is less than 200 ppm.

50. The method of claim 42 wherein the WEF is less than 180 ppm.

51. The method of claim 42 wherein the TF is greater than 2000 ppm.

52. The method of claim 42 wherein the TF is greater than 2500 ppm.

53. The method of claim 42 wherein the TF is less than 4500 ppm.

54. The method of claim 42 wherein the fluoride-mineralized carrier has a water absorption of at least 50 weight percent.

55. The method of claim 42 wherein the fluoride-mineralized carrier has a silica content less than 0.05 weight percent.

56. The method of claim 42 wherein the fluoride-mineralized carrier has a magnesia content less than 0.05 weight percent.

57. The method of claim 42 wherein the fluoride-mineralized carrier has a surface area of from 0.5 m²/g to 5 m²/g.

58. The method of claim 42 wherein the fluoride-mineralized carrier has a surface area of from 0.7 m²/g to 3 m²/g.

59. The method of claim 42 wherein the fluoride-mineralized carrier is multi-lobed.

60. The method of claim 42 wherein the ethylene epoxidation catalyst further comprises an alkali metal promoter selected from the group consisting of lithium, sodium, potassium, rubidium, cesium, and a combination thereof.

61. The method of claim 60 wherein the ethylene epoxidation catalyst further comprises:
a first co-promoter selected from the group consisting of sulfur, phosphorus, boron, and a combination thereof; and
a second co-promoter selected from the group consisting of tungsten, molybdenum, chromium, and a combination thereof.

62. The method of claim 61 wherein the ethylene epoxidation catalyst further comprises a further metal selected from the group consisting of beryllium, magnesium, calcium, strontium, barium, titanium, hafnium, zirconium, vanadium, thallium, thorium, tantalum, niobium, gallium, germanium, manganese, and a combination thereof.
